SEQUENCE LISTING (4 sequences)

SEQ ID NO: 1 - DNA, 11 bp, Homo sapiens
  source 1..11: mol_type = genomic DNA
tcacggcaaca

SEQ ID NO: 2 - protein, 25 aa, Homo sapiens
LDGGTLSRQHNCGTPLPVSSEKDMI

SEQ ID NO: 3 - DNA, 10 bp, synthetic construct
  source 1..10: mol_type = other DNA
tcacgcaaca

SEQ ID NO: 4 - protein, 24 aa, synthetic construct
LDGGTLSRNIIVAHHCLFHLRKIX

(19) United States
(12) Patent Application Publication
(10) Pub. No.: US 2026/0272923 A1
(43) Pub. Date: Sep. 17, 2026

(54) NONSENSE MEDIATED DECAY INHIBITOR COMPOUNDS

(71) Applicant: The Johns Hopkins University, Baltimore, MD (US)

(72) Inventors: Nicolas Wyhs, San Diego, CA (US); Ashley L. Cook, Springfield, PA (US); Surojit Sur, Ellicott City, MD (US); Bert Vogelstein, Baltimore, MD (US); Nickolas Papadopoulos, Towson, MD (US); Shibin Zhou, Owings Mills, MD (US); Kenneth W. Kinzler, Frankford, DE (US)

(21) Appl. No.: 19/164,764

(22) PCT Filed: Mar. 13, 2024
(86) PCT No.: PCT/US2024/019788

§ 371 Date: Sep. 12, 2025

Related U.S. Application Data
(60) Provisional application No. 63/451,738, filed on Mar. 13, 2023.

(51) Int. Cl. A61K 31/506; A61K 45/06; A61P 35/00; C07D 401/04; C07D 401/14; C07K 14/47; C12N 9/10; C12N 9/14; C12N 15/67

(52) U.S. Cl. CPC A61K 31/506; A61K 45/06; A61P 35/00; C07D 401/04; C07D 401/14; C07K 14/4746; C12N 9/1029; C12N 9/14; C12N 15/67; C12Y 203/01; C12Y 306/05002

(57) ABSTRACT

Provided herein are compounds, pharmaceutical compositions, and methods for nonsense mediated decay (NMD) inhibition and cancer treatment.

### CROSS REFERENCE TO RELATED APPLICATIONS

This application claims the benefit of U.S. Provisional Application No. 63/451,738, filed on Mar. 13, 2023, which is incorporated herein by reference in its entirety.

### STATEMENT REGARDING FEDERALLY SPONSORED RESEARCH OR DEVELOPMENT

This invention was made with government support under grant numbers CA006973 and CA270403, awarded by the National Institutes of Health. The government has certain rights in the invention.

### TECHNICAL FIELD

Described herein are compounds, pharmaceutical compositions, and methods for nonsense mediated decay (NMD) inhibition and cancer treatment.

### BACKGROUND

Despite great success with cancer immunotherapies, approved immunotherapies are not available for most cancer patients and less than half of patients who are treated will realize a durable response with immunotherapy alone. Studies have focused on identifying patients most likely to benefit from immunotherapy and discovering novel combinations to increase the number of targetable cancer associated neoantigens. Furthermore, studies correlate tumor insertion and deletion (indel) mutation load with immunotherapeutic response but not all tumors with high indel mutational loads respond to checkpoint inhibitors. There is a clear need to identify mechanisms of immune surveillance escape for these patients and leverage this knowledge to maximize the presented mutational load.

The typical adult solid tumor contains somatic mutations, many of which have the potential to create novel neoantigens or Mutation Associated NeoAntigens (MANA). Some of these mutation are insertions/deletions (indels) or alter splicing that alter the open reading frame (ORF) of the transcript. This subset is of particular interest in terms of MANA because they can result in long peptides that are foreign to healthy cells. The majority of driver gene mutations result in loss of protein or function. These alterations often occur in tumor suppressor genes and can present as indel mutations. Unfortunately, both of these alterations can also trigger the mRNA quality control mechanism: nonsense mediated decay (NMD). In normal cells. NMD plays an important role in messenger RNA (mRNA) quality control, as well as normal gene expression. In cancer cells, however, it may assist with immunoevasion by downregulating mutant RNA transcripts. This prevents translation of these mutant proteins, stopping their presentation via MHC class I proteins to host immune cells. It has been shown that NMD pathway knockdown in vitro and in vivo causes an increased anti-tumor immune response. Analysis of patient samples from The Cancer Genome Atlas (TCGA) also shows that disruption ofNMD factors correlates to an increase number of mutant specific proteins. Despite studies showing little toxicity with NMD inhibition, there is a need of a specific inhibitor of the pathway with good bioavailability.

### SUMMARY

Provided herein are compounds of Formula I or a pharmaceutically acceptable salt thereof.

Also provided herein are pharmaceutical compositions comprising: (a) the compound of Formula I or a pharmaceutically acceptable salt thereof; and (b) a pharmaceutically acceptable carrier. In some embodiments, the compound of Formula I inhibits nonsense mediated decay (NMD). In some embodiments, the cell-surface expression of a neoantigen is increased. In some embodiments, the cell-surface expression is presentation of the neoantigen on a MHC class I HLA molecule. In some embodiments, the neoantigen is derived from a TP53 (tumor protein p53) gene, EXOC1 gene. SPTAN1 gene, RAB14 gene, or ZDHHC16 gene.

Also provided herein are methods of inhibiting nonsense mediated decay (NMD), the method comprising delivering the compound of Formula I into a cell, thereby inhibiting NMD in the cell. In some embodiments, the cell-surface expression of a neoantigen is increased. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHCI6 gene.

Also provided herein are methods of enhancing immunotherapy in a subject in need thereof, the method comprising administering to the subject any one of the compounds, or any one of the pharmaceutical compositions described herein. In some embodiments, any one of the compounds, or any one of the pharmaceutical compositions described herein inhibits nonsense mediated decay (NMD). In some embodiments, cell-surface expression of a neoantigen is increased in the subject following administration. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHC16 gene.

In some embodiments, the subject has been identified or diagnosed as having a cancer. In some embodiments, the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma. B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

Also provided herein are methods of treating cancer in a subject in need thereof, the method comprising administering to the subject: (a) any one of the compounds, or any one of the pharmaceutical compositions described herein; and (b) an immunotherapy. In some embodiments, the immunotherapy comprises an immune checkpoint inhibitor, an oncolytic virus therapy, a cell-based therapy, and a cancer vaccine. In some embodiments, the method further comprises administering to the subject one or more additional anticancer therapies. In some embodiments, the one or more additional anticancer therapies comprise a chemotherapeutic agent, ionizing radiation, a therapeutic antibody, or gene therapy.

In some embodiments, any one of the compounds, or any one of the pharmaceutical compositions described herein inhibits nonsense mediated decay (NMD). In some embodiments, the cell-surface expression of a neoantigen is increased in the subject following administration. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene. RAB14 gene, or ZDHHC16 gene.

In some embodiments, the cancer is a solid tumor, microsatellite-instability high (MSI-high) tumor, hematological tumor, sarcoma, osteosarcoma, glioblastoma, neuroblastoma, melanoma, rhabdomyosarcoma. Ewing sarcoma, osteosarcoma. B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

Also provided herein are compounds of Formula II or a pharmaceutically acceptable salt thereof.

Also provided herein are pharmaceutical compositions comprising: (a) the compound of Formula II or a pharmaceutically acceptable salt thereof; and (b) a pharmaceutically acceptable carrier. In some embodiments, the compound of Formula II inhibits nonsense mediated decay (NMD). In some embodiments, the cell-surface expression of a neoantigen is increased. In some embodiments, the cell-surface expression is presentation of the neoantigen on a MHC class I HLA molecule. In some embodiments, the neoantigen is derived from a TP53 (tumor protein p53) gene, EXOC1 gene. SPTAN1 gene, RAB14 gene, or ZDHHC16 gene.

Also provided herein are methods of inhibiting nonsense mediated decay (NMD), the method comprising delivering the compound of Formula II into a cell, thereby inhibiting NMD in the cell. In some embodiments, the cell-surface expression of a neoantigen is increased. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHC16 gene.

Also provided herein are methods of enhancing immunotherapy in a subject in need thereof, the method comprising administering to the subject any one of the compounds, or any one of the pharmaceutical compositions described herein. In some embodiments, any one of the compounds, or any one of the pharmaceutical compositions described herein inhibits nonsense mediated decay (NMD). In some embodiments, cell-surface expression of a neoantigen is increased in the subject following administration. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHC16 gene.

In some embodiments, the subject has been identified or diagnosed as having a cancer. In some embodiments, the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma. B-cell lymphoma, B-cell non-Hodgkin's lymphoma. Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

Also provided herein are methods of treating cancer in a subject in need thereof, the method comprising administering to the subject: (a) any one of the compounds, or any one of the pharmaceutical compositions described herein; and (b) an immunotherapy. In some embodiments, the immunotherapy comprises an immune checkpoint inhibitor, an oncolytic virus therapy, a cell-based therapy, and a cancer vaccine. In some embodiments, the method further comprises administering to the subject one or more additional anticancer therapies. In some embodiments, the one or more additional anticancer therapies comprise a chemotherapeutic agent, ionizing radiation, a therapeutic antibody, a ribosome targeting read through agent (e.g., ELX02, PTC124), or gene therapy.

In some embodiments, any one of the compounds, or any one of the pharmaceutical compositions described herein inhibits nonsense mediated decay (NMD). In some embodiments, the cell-surface expression of a neoantigen is increased in the subject following administration. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene. RAB14 gene, or ZDHHC16 gene.

In some embodiments, the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma. B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

Also provided herein are compounds of Formula III or a pharmaceutically acceptable salt thereof.

Also provided herein are pharmaceutical compositions comprising: (a) the compound of Formula III or a pharmaceutically acceptable salt thereof; and (b) a pharmaceutically acceptable carrier. In some embodiments, the compound of Formula III inhibits nonsense mediated decay (NMD). In some embodiments, the cell-surface expression of a neoantigen is increased. In some embodiments, the cell-surface expression is presentation of the neoantigen on a MHC class I HLA molecule. In some embodiments, the neoantigen is derived from a TP53 (tumor protein p53) gene, EXOC1 gene. SPTAN1 gene, RAB14 gene, or ZDHHC16 gene.

Also provided herein are methods of inhibiting nonsense mediated decay (NMD), the method comprising delivering the compound of Formula III into a cell, thereby inhibiting NMD in the cell. In some embodiments, the cell-surface expression of a neoantigen is increased. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHC16 gene.

Also provided herein are methods of enhancing immunotherapy in a subject in need thereof, the method comprising administering to the subject any one of the compounds, or any one of the pharmaceutical compositions described herein. In some embodiments, any one of the compounds, or any one of the pharmaceutical compositions inhibits nonsense mediated decay (NMD). In some embodiments, cell-surface expression of a neoantigen is increased in the subject following administration. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHC16 gene.

In some embodiments, the subject has been identified or diagnosed as having a cancer. In some embodiments, the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

Also provided herein are methods of treating cancer in a subject in need thereof, the method comprising administering to the subject: (a) any one of the compounds, or any one of the pharmaceutical compositions described herein; and (b) an immunotherapy. In some embodiments, the immunotherapy comprises an immune checkpoint inhibitor, an oncolytic virus therapy, a cell-based therapy, and a cancer vaccine. In some embodiments, the method further comprises administering to the subject one or more additional anticancer therapies. In some embodiments, the one or more additional anticancer therapies comprise a chemotherapeutic agent, ionizing radiation, a therapeutic antibody, or gene therapy.

In some embodiments, any one of the compounds, or any one of the pharmaceutical compositions described herein inhibits nonsense mediated decay (NMD). In some embodiments, the cell-surface expression of a neoantigen is increased in the subject following administration. In some embodiments, the cell-surface expression is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHC16 gene.

In some embodiments, the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

In one aspect, this disclosure features compounds having formula (IV):

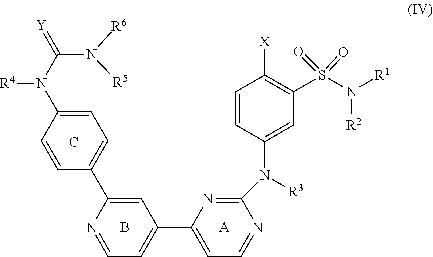

or a pharmaceutically acceptable salt thereof; wherein:
each occurrence of R¹ and R² is independently selected from the group consisting of: H and C₁₋₆ alkyl, which is optionally substituted with —OH, —SH, F, or —NH₂; or
R¹ and R², taken together with the nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of: N, N(R⁷), O, and S; and wherein the ring is optionally substituted with 1-4 independently selected R^{a};
wherein R⁷ is H or C₁₋₆ alkyl, which is optionally substituted with —OH, —SH, —F, and —NH₂;
each occurrence of R³ and R⁴ is independently selected from the group consisting of: H and C₁₋₃ alkyl;
each occurrence of R⁵ and R⁶ is independently selected from the group consisting of: H and C₁₋₆ alkyl,
X is H, —F, Cl or CN;
Y is O or S;
each of rings A, B, and C is independently and optionally substituted with 1-4 independently selected R^{a};
each occurrence of R^{a} is independently selected from the group consisting of halo, C₁₋₆ alkyl, C₁₋₆ haloalkyl, cyano, C₁₋₆ alkoxy, C₁₋₆ haloalkoxy, C₁₋₆ thioalkoxy, C₁₋₆ halothioalkoxy, S(O)₂(C₁₋₆ alkyl), C₃₋₆ cycloalkyl, C₆₋₁₀ aryl, NR⁸R⁹, C(O)R⁸, and C(O)NR⁸R⁹, and
each occurrence of R⁸ and R⁹ is independently selected from the group consisting of: H and C₁₋₃ alkyl.

In some embodiments, each occurrence of R¹ and R² is independently selected from the group consisting of: H and C₁₋₆ alkyl, which is optionally substituted with —OH, —SH, F, or —NH₂.

In certain embodiments, each occurrence of R¹ and R² is an independently selected C₁₋₆ alkyl, which is optionally substituted with —OH, —SH, F, or —NH₂.

In certain embodiments, each occurrence of R¹ and R² is an independently selected C₁₋₆ alkyl (e.g., C₁₋₃ alkyl), which is substituted with —OH, —SH, F, or —NH₂ (e.g., substituted with —OH). For example, each occurrence of R¹ and R² can be —CH₂CH₂OH.

In some embodiments. R¹ and R², taken together with the nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of N, N(R⁷), O, and S; and wherein the ring is optionally substituted with 1-4 independently selected R^{a}.

In certain embodiments, R¹ and R², taken together with the nitrogen atom connecting them, forms a saturated ring including 4-8 ring atoms, wherein from 0-2 ring atoms (in addition to the nitrogen atom connecting R¹ and R²) are ring heteroatoms each independently selected from the group consisting of N(R⁷), O, and S; and wherein the ring is optionally substituted with 1-4 independently selected R^{a}.

In certain embodiments, R¹ and R², taken together with the nitrogen atom connecting them, forms a saturated ring including 4-8 ring atoms, wherein from 0-2 ring atoms (in addition to the nitrogen atom connecting R¹ and R²) are each an independently selected N(R⁷); and wherein the ring is optionally substituted with 1-4 independently selected R^{a}.

In certain embodiments, R¹ and R², taken together with the nitrogen atom connecting them, forms a saturated ring including 4-8 ring atoms, wherein 1 ring atom (in addition to the nitrogen atom connecting R¹ and R²) is N(R⁷); and wherein the ring is optionally substituted with 1-4 independently selected R^{a}.

In certain embodiments, R¹ and R², taken together with the nitrogen atom connecting them, forms a saturated ring including 6 ring atoms, wherein 1 ring atom (in addition to the nitrogen atom connecting R¹ and R²) is N(R⁷); and wherein the ring is optionally substituted with 1-4 independently selected R^{a}.

In certain embodiments, the ring carbon atoms are unsubstituted.

In certain embodiments, R⁷ is H.

In certain embodiments, R⁷ is C₁₋₆ alkyl, which is optionally substituted with —OH, —SH, —F, and —NH₂.

In certain embodiments, R⁷ is C₁₋₆ alkyl. For example, R⁷ can be C₁₋₃ alkyl (e.g., CH₃).

In certain embodiments, each occurrence of R³ and R⁴ is H.

In certain embodiments, one of R⁵ and R⁶ is H, and the other of R⁵ and R⁶ is C₁₋₆ alkyl.

In certain of these embodiments, one of R⁵ and R⁶ is H, and the other of R⁵ and R⁶ is C₁₋₃ alkyl. For example, one of R⁵ and R⁶ can be H, and the other of R⁵ and R⁶ can be CH₃.

In certain embodiments, X is Cl.

In certain embodiments, Y is O.

In certain embodiments, each of rings A, B, and C is unsubstituted.

In another aspect, this disclosure features compounds having formula (V):

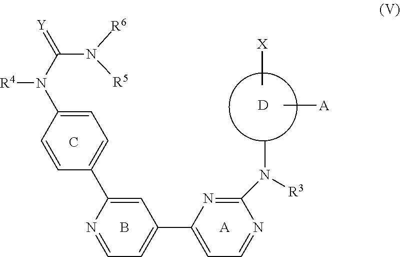

or a pharmaceutically acceptable salt thereof; wherein:
ring D is
   phenyl;
   heteroaryl of 5-6 ring atoms, wherein 1-3 ring atoms are heteroatoms, each independently selected from the group consisting of N, N(R⁷), O, and S; and wherein R⁷ is H, C₁₋₃ alkyl, or —C(O)C₁₋₃ alkyl; or
   heterocyclyl or heterocycloalkenyl of 4-7 ring atoms, wherein from 1-3 ring atoms are heteroatoms, each independently selected from the group consisting of N, N(R⁷), O, and S; and wherein R⁷ is H, C₁₋₃ alkyl, or C(O)C₁₋₃ alkyl;
X is H, —F, Cl, C₁-C₄ alkyl, or CN;
A is —SO₂NR¹R², —C(O)NR⁸R², —SO₃H, —SO₂R^{A}, or —PO(OH)₂;
R^{A} is C₁₋₆ alkyl, which is optionally substituted with 1-3 substituents independently selected from the group consisting of: —OH, C₁₋₄ alkoxy, C₁₋₄ haloalkoxy, and F;
each occurrence of R¹ and R² is independently selected from the group consisting of: H and C₁₋₆ alkyl, wherein the C₁₋₆ alkyl is optionally substituted with 1-3 substituents independently selected from the group consisting of:
   —CO₂H;
   —CO₂(C₁₋₄ alkyl);
   —OC(O)(CH₂)ₙ—CO₂H, wherein n is 1-4 (e.g., n is 2);
   —OC(O)(CH₂)ₙ—CO₂(C₁₋₄ alkyl), wherein n is 1-4 (e.g., n is 2);
   —OH;
   C₁₋₄ alkoxy;
   C₁₋₄ haloalkoxy;
   —F, and
   —NR′R″, wherein each of R′ and R′ is independently selected from the group consisting of H and C₁₋₄ alkyl;
or
R¹ and R², taken together with the nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of: N, N(R⁷), O, and S; and wherein the ring is optionally substituted with 1-4 independently selected R^{a}; wherein R⁷ is H, C₁₋₆ alkyl, or —C(O)C₁₋₆ alkyl;
each of rings A, B. and C is independently and optionally substituted with 1-3 independently selected R^{a};
each occurrence of R³ and R⁴ is independently selected from the group consisting of: H and C₁₋₃ alkyl;
each occurrence of R⁵ and R⁶ is independently selected from the group consisting of: H and C₁₋₆ alkyl,
Y is O or S;
each occurrence of R^{a} is independently selected from the group consisting of: halo, C₁₋₆ alkyl, C₁₋₆ haloalkyl, cyano, C₁₋₆ alkoxy, C₁₋₆ haloalkoxy, C₁₋₆ thioalkoxy, C₁₋₆ halothioalkoxy, S(O)₂(C₁₋₆ alkyl), C₃₋₆ cycloalkyl, C₆₋₁₀ aryl, NR⁸R⁹, C(O)R¹, and C(O)NR⁸R⁹, and
each occurrence of R⁸ and R⁹ is independently selected from the group consisting of: H and C₁₋₃ alkyl.

In some embodiments, ring D is phenyl.

In certain embodiments, ring D has the formula:

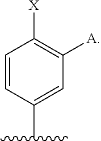

In some embodiments, ring D is heteroaryl of 5-6 ring atoms, wherein from 1-3 ring atoms are heteroatoms, each independently selected from the group consisting of N, N(R⁷), O, and S; and wherein R⁷ is H. C₁₋₃ alkyl, or —C(O)C₁₋₃ alkyl.

In certain embodiments, ring D is heteroaryl of 5 ring atoms, wherein from 1-3 ring atoms are heteroatoms, each independently selected from the group consisting of N, N(R⁷), O, and S; and wherein R⁷ is H, C₁₋₃ alkyl, or —C(O)C₁₋₃ alkyl.

In certain embodiments, ring D is 2H-pyrrolyl.

For example, ring D can be:

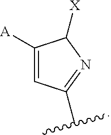

In certain embodiments, ring D is heteroaryl of 6 ring atoms, wherein from 1-3 ring atoms are heteroatoms, each independently selected from the group consisting of N, N(R⁷), O, and S; and wherein R⁷ is H, C₁₋₃ alkyl, or —C(O)C₁₋₃ alkyl.

In certain embodiments, ring D is pyridyl.

In certain embodiments, ring D is:

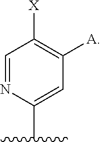

In certain embodiments, A is —SO₂NR¹R².

In certain of these embodiments, each occurrence of R¹ and R² is H.

In other embodiments, each occurrence of R¹ and R² is C₁₋₆ alkyl that is optionally substituted.

In still other embodiments. R¹ and R², taken together with the nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of N, N(R⁷), O, and S; and wherein the ring is optionally substituted with 1-4 independently selected R^{a}; wherein R⁷ is H, C₁₋₆ alkyl, or —C(O)C₁₋₆ alkyl.

In certain embodiments, X is Cl.

The term “alkyl,” as used herein, refers to a saturated hydrocarbon chain that is a straight chain or branched chain, containing the indicated number of carbon atoms. For example, C₁-C₄ alkyl group indicates that the group has from 1 to 4 (inclusive) carbon atoms in it. Similarly, C₁-C₁₀ alkyl group indicates that the group has from 1 to 10 (inclusive) carbon atoms in it. Unless stated otherwise specifically in the specification, an alkyl group is optionally substituted. The term “haloalkyl”, as used herein, refers to an alkyl, in which one or more hydrogen atoms is/are replaced with an independently selected halo.

The term “cycloalkyl” or “carbocyclic ring”, as used herein, refers to a stable non-aromatic monocyclic or polycyclic hydrocarbon radical consisting solely of carbon and hydrogen atoms, which may include fused or bridged ring systems, having from three to fifteen carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl.

The term “aryl”, as used herein, refers to a hydrocarbon ring system radical comprising, e.g., 6 to 18 carbon atoms and at least one aromatic ring. The aryl radical can be a monocyclic, bicyclic, tricyclic, or tetracyclic ring system, which may include fused or bridged ring systems. Aryl radicals can include, but not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

Also provided herein are pharmaceutical compositions comprising: (a) the compound of Formula IV or a pharmaceutically acceptable salt thereof; and (b) a pharmaceutically acceptable carrier as well as methods of using the same as described anywhere herein.

Also provided herein are pharmaceutical compositions comprising: (a) the compound of Formula V or a pharmaceutically acceptable salt thereof; and (b) a pharmaceutically acceptable carrier as well as methods of using the same as described anywhere herein.

### BRIEF DESCRIPTION OF DRAWINGS

FIG. 1A shows an exemplary schematic of high throughput screen used to identify inhibitors of NMD. Mutant transcripts are represented with a smaller size in the cartoon for illustrative purposes only.

FIG. 1B shows mutant RNA reads relative to wild type reads for the top 30 hits from the HTS. The dotted line represents the minimum fraction required to be considered a hit (>4 standard deviations above DMSO control).

FIG. 1C shows targeted RNA sequencing results of isogenic RPTec knockout clones treated with the 8 best hits from the HTS.

FIG. 1D shows TP53 western blot on an RPE TP53 knockout clone using the four hits that validated in RPTec isogenic lines.

FIG. 1E shows Western blot analysis of full-length and TP53β after treatment with two NMD inhibition lead candidates. TP53β as well as mutant TP53 are prominently induced by LY3023414 whereas full-length is not. Note that RPE TP53 clone 223 is a heterozygous knockout with one wt allele intact where as RPTec TP53 KO 588 is homozygous.

FIG. 1F shows Real time PCR showing LY3023414 causes increased expression of the NMD controlled alternative transcript for TP53 (Beta) in parent cell lines for RPE and RPTec.

FIG. 2A shows mutant transcript recovery rates for genes containing heterozygous indel mutations based on RNA-sequencing results in cell lines treated with LY3023414. Strict inclusion criteria were used, so only mutations with sufficient sequencing coverage are shown.

FIG. 2B shows targeted high coverage RNA-sequencing confirms recovery of mutant transcript levels in NCI-H358 and LS180 cancer cell lines treated with LY3023414. RNF43 and DROSHA serve as controls (common heterozygous SNP's).

FIG. 2C shows Western blot analyses on NCI-H358 cells showing mutant and wild type protein expression in SPTAN1 and EXOC1. The C-terminal SPTAN1 antibody is downstream of the out of frame indel mutation and is not expected to identify the mutant allele.

FIG. 2D shows RNA transcript levels in NCI-H358 and LS180 xenografts treated by oral gavage with LY3023414 and assayed after 24 hours. Student T-Test for target genes are all p<0.05, while the null hypothesis holds for RNF43 (common SNP).

FIG. 3A shows change in mutant allele expression in genes with heterozygous indels known to be sensitive to NMD inhibition after siRNA knockdown was performed on kinases targeted by LY3023414. RNF43 and DROSHA are common SNP's and serve as a controls.

FIG. 3B shows targeted RNA-sequencing known NMD targets and controls in cells treated with SMG1 specific inhibitor restores expression of NMD down regulated transcripts.

FIG. 3C shows Western blot on genes with heterozygous indels treated with LY3023414 or an SMG1 specific small molecule inhibitor demonstrating restored expression of proteins down regulated by NMD.

FIG. 3D shows Western blot evaluating phosphorylation status of UPF1 in cells treated with D-DMSO, L-LY3023414 or S-SMG1 specific small molecule inhibitor.

FIG. 4A shows the structure of novel NMD inhibitor compound of Formula I.

FIG. 4B shows Kinativ™ Assay results for the compound of Formula I at 100 nM run in duplicate.

FIG. 4C shows targeted RNA-sequencing on three genes with heterozygous out of frame indel mutations in LS180 cancer cells treated in a dose response with the compound of Formula I. RNF43 serves as a control (common heterozygous SNP).

FIG. 4D shows Western blot on EXOC1 in NCI-H358 cells treated for 24 hours.

FIG. 4E shows phosphorylated UPFJ western on three cell lines treated with the compound of Formula I, SMG1 inhibitor, or DMSO.

FIG. 4F shows fold change in mutant transcript levels of NMD controlled genes containing heterozygous out of frame indel mutations in H358 Cells placed subcutaneously in a NOD-SCID mice and treated once with IP injection of vehicle or the compound of Formula I.

FIG. 5A shows DNA and protein sequence from heterozygous mutation containing EXOC1 gene in NCI-H358. Red color indicate mutant specific residues.

FIGS. 5B-5C shows MHC class I HLA presentation of mutant specific peptide sequences from NCI-H358 (FIG. 5B) and LS180 (FIG. 5C) cells by quantitative HPLC-Mass Spectrometry. Note Y-axis scale changes between some samples.

FIG. 5D shows TP53 gene structure and mutant DNA sequence for NCI-H716 and NCI-H2228. Capital letters represent exonic sequence: lowercase letters represent intronic sequence. The orange colored base indicates the mutation.

FIG. 5E shows Western blot against TP53 in the presence or absence of inhibitor in NCI-H716 and H2228 cell lines.

FIG. 5F shows interferon gamma levels based on ELISA in a co-culture assay with NCI-H716 and H2228 cells, NMD inhibitor, human CD8+ T-cells, and bispecific antibody for TP53 and CD3. Chemotherapies 5-FU and etoposide are shown as controls.

FIG. 5G shows cell killing based on luciferase levels in a co-culture assay in NCI-H716 cells with and without A24 expression, treated with TP53-CD3 bispecific antibody, NMD inhibitor and human CD8+ T-cells.

FIG. 6A shows an exemplary treatment schedule for syngeneic tumor mouse treatment.

FIGS. 6B-6C show average (n=8) (FIG. 6B) and Spider plot of LLC tumor size (FIG. 6C) in C57BL/6N mice treated with 30 mg/kg of the compound of Formula I or vehicle control. Error bars show 95% confidence intervals in all plots. Difference is statistically significant based on one-way ANOVA with Dunnett's post test.

FIGS. 6D-6E show average (n=8) (FIG. 6D) and Spider plot of RENCA tumor size (FIG. 6E) in BALB/C mice treated with 20 mg/kg of the compound of Formula I or vehicle control. Difference is statistically significant based on one-way ANOVA with Dunnett's post test FIGS. 6F-6G show average (n=6 or 8) (FIG. 6F) and spider plot (FIG. 6G) of immunodeficient C57BL/6N SCID mice treated with the same schedule shown in FIG. 6A.

FIGS. 6H-6I show average (n=6 or 8) (FIG. 6H) and spider plot (FIG. 6I) of immunodeficient NSG (BALB/c) mice treated with the same schedule shown in FIG. 6A.

FIG. 7A shows RPE STAG2 knockout cell lines that were treated with the compound of Formula II re-expresses mutant RNA.

FIG. 7B shows RPE STAG2 knockout cell lines that were treated with the compound of Formula II re-expresses mutant protein.

FIG. 8A shows the compound of Formula III re-expresses mutant RNA in vitro.

FIG. 8B shows the compound of Formula III re-expresses mutant protein in vitro.

FIGS. 9A-9B show the compound of Formula III having in vivo NMD inhibition activity. Nude mice with subcutaneous tumors (either H358 (FIG. 9A) or LS180 (FIG. 9B)) treated with IP dose at 30 mg/kg of the compound of Formula III. Data is targeted RNA-sequencing showing re-expression of numerous genes whose RNA is downregulated by NMD due to out of frame insertion or deletion mutations.

### DETAILED DESCRIPTION

Nonsense mediated decay (NMD) is a cellular quality control pathway that degrades aberrant RNA transcripts containing premature stop codons. A subset of driver and passenger mutations in cancer cells trigger NMD and the subsequent destruction of these mutant RNAs can potentially conceal neoantigens and bolsters immunoevasion. Recent advances have highlighted the power of cancer immunotherapy but its broad-based application is limited by the number of targetable antigens on the cell surface, wherein disruption of the nonsense mediated decay pathway by inhibiting the kinase SMG1 can cause presentation of T-cell targetable cancer specific antigens on the cell surface.

Provided herein are compounds or derivatives thereof of Formula I, or a pharmaceutically acceptable salt thereof.

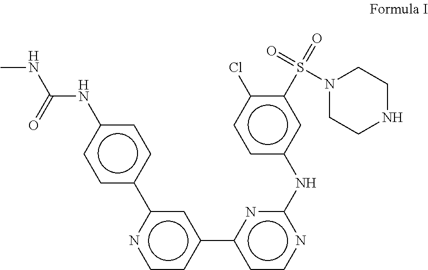

Also provided herein are pharmaceutical compositions that include (a) the compound of Formula I or a pharmaceutically acceptable salt thereof; and (b) a pharmaceutically acceptable carrier.

Also provided herein are compounds or derivatives thereof of Formula II or a pharmaceutically acceptable salt thereof.

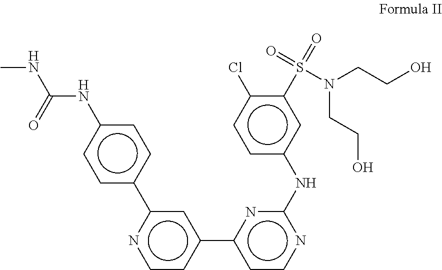

Also provided herein are pharmaceutical compositions that include (a) the compound of Formula II or a pharmaceutically acceptable salt thereof; and (b) a pharmaceutically acceptable carrier.

Also provided herein are compounds or derivatives thereof of Formula III or a pharmaceutically acceptable salt thereof.

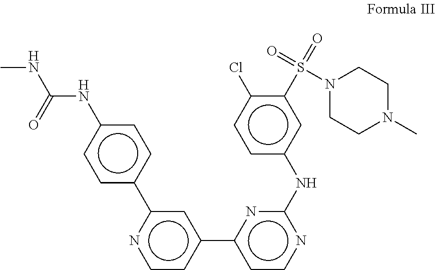

Also provided herein are pharmaceutical compositions that include (a) the compound of Formula III or a pharmaceutically acceptable salt thereof; and (b) a pharmaceutically acceptable carrier.

Also provided herein are methods of inhibiting nonsense mediated decay (NMD) that include delivering a compound of Formula I, Formula II, or Formula III, or combinations thereof, into a cell. Also provided herein are methods of enhancing immunotherapy and/or treating cancer in a subject in need thereof, the method including administering to the subject any one or more of the compounds or the pharmaceutical compositions described herein.

Various non-limiting aspects of these methods are described herein, and can be used in any combination without limitation.

It must be noted that, as used in the specification and the appended claims, the singular forms “a,” “an” and “the” include plural referents unless the context clearly dictates otherwise.

As used herein, the term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%. 6%, 5%, 4%, 3%, 2%, 1%, or less of the referred value.

As used herein, the terms “cancer”. “malignancy”, “neoplasm”, “tumor”, and “carcinoma”, refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

As used herein, a “cell” can refer to either a prokaryotic or eukaryotic cell, optionally obtained from a subject or a commercially available source.

As used herein, an “effective amount” or a “therapeutically effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease. A “therapeutically effective amount.” as used herein, refers to that amount of the therapeutic agent sufficient to ameliorate the disorder. For example, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

As used herein, a “patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the terms “treating”, or “treatment” can refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) can also broadly include any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In some embodiments, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

### A. Nonsense Mediated Decay (NMD)

Nonsense mediated decay (NMD) is a conserved surveillance pathway and translation-coupled mechanism, wherein its main function is to reduce errors in gene expression by eliminating mRNAs containing premature translation-termination codons (PTCs). Thus NMD functions in cells to ensure the quality of transcripts and to modulate transcript abundance. NMD has three main factors which include UPF1, UPF2 and UPF3 (UPF3A and UPF3B in humans), that make up the conserved core of the NMD pathway. In mammals, UPF2 and UPF3 are part of the exon-exon junction complex (EJC) bound to mRNA after splicing along with other proteins, eIF4AIII. MLN51, and the Y14/MAGOH heterodimer, which also function in NMD. Furthermore, UPF1 phosphorylation is controlled by the proteins SMG-1, SMG-5, SMG-6 and SMG-7.

The role of NMD in cancer development is complex, wherein, in some cases, tumors have exploited NMD to downregulate gene expression by apparently selecting for mutations causing destruction of key tumor-suppressor mRNAs. In other cases, tumors adjust NMD activity to adapt to their microenvironment which could provide an opportunity for therapeutic intervention as NMD inhibition has been shown to lead to the production of neoantigens that stimulate an immune system attack on tumors.

Neoantigens are cancer-associated proteins that can trigger anti-cancer immune responses, wherein cancer cells harbor clonal mutations that cause them to produce these unique proteins which are not present in normal cells. In some cases, a general goal of cancer immunotherapy is to enhance such responses so that the immune system can eliminate neoantigen-expressing cancer cells. Neoantigens are presented to neighboring immune cells in a multistep process that involves degradation of the mutant protein by the proteasome and the binding of the resultant peptides to the class 1 major histocompatibility complex (MHC class 1). The bound peptide/MHC complexes localize to the cell surface where they can engage T cells via the T cell receptor. This interaction allows the adaptive immune system to recognize the abnormal proteins produced by tumors and to initiate robust cytotoxic responses that ultimately prevent disease progression.

Mutation of TP53 (tumor protein p53, commonly referred to as p53) can create neoantigen, present at high frequency in many types of cancer. TP53 was among the first tumor suppressor genes identified and is inactivated in the majority of human tumors. In some embodiments, a neoantigen can be derived from a TP53 mutation. Mutations in TP53 most commonly occur as single-nucleotide variants at positions that cluster in the DNA-binding domain. Proteins encoded by the common mutant TP53 alleles are expressed at elevated levels, wherein these overexpressed proteins, unique to tumor cells, are potentially immunogenic and therefore represent an attractive target for immunotherapies.

In some embodiments, the neoantigen can be derived from a gene with a nonsense mutation. In some embodiments, the neoantigen can be derived from a gene with an indel mutation, wherein the indel mutation results in a frameshift. In some embodiments, the neoantigen can be derived from a gene with a splicing mutation, wherein the splicing mutation results in a frameshift. In some embodiments, the neoantigen can be derived from a TP53 gene. In some embodiments, the neoantigen can be derived from an EXOC1 gene. In some embodiments, the neoantigen can be derived from a SPTAN1 gene. In some embodiments, the neoantigen can be derived from a RAB14 gene. In some embodiments, the neoantigen can be derived from a ZDHHC16 gene.

### B. Compounds

Provided herein are compounds or derivatives thereof. In some embodiments, a compound provided herein is a compound of Formula I:

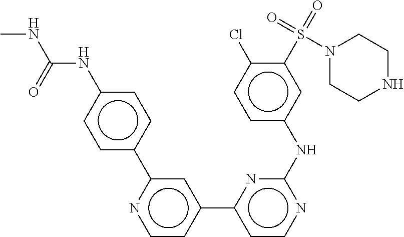

or a pharmaceutically acceptable salt thereof.

In some embodiments, the compound of Formula I or a pharmaceutically acceptable salt thereof can be used to inhibit nonsense mediated decay (NMD). In some embodiments, the compound of Formula I or a pharmaceutically acceptable salt thereof can be useful as a small molecule inhibitor of Nonsense Mediated mRNA Decay Associated PI3K Related Kinase (SMG-1). In some embodiments, the compound of Formula I can be referred to as KVS0001.

In some embodiments, a compound provided herein is a compound of Formula II:

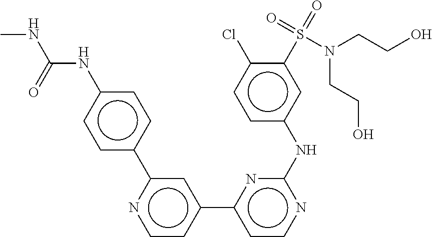

or a pharmaceutically acceptable salt thereof.

In some embodiments, the compound of Formula II or a pharmaceutically acceptable salt thereof can be used to inhibit nonsense mediated decay (NMD). In some embodiments, the compound of Formula II or a pharmaceutically acceptable salt thereof can be useful as a small molecule inhibitor of Nonsense Mediated mRNA Decay Associated PI3K Related Kinase (SMG-1). In some embodiments, the compound of Formula II can be referred to as KVS0002.

In some embodiments, a compound provided herein is a compound of Formula III:

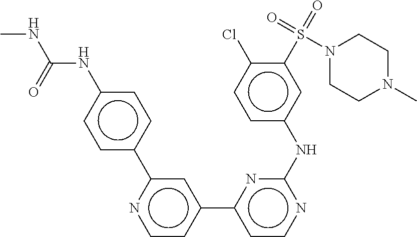

or a pharmaceutically acceptable salt thereof.

In some embodiments, the compound of Formula III or a pharmaceutically acceptable salt thereof can be used to inhibit nonsense mediated decay (NMD). In some embodiments, the compound of Formula III or a pharmaceutically acceptable salt thereof can be useful as a small molecule inhibitor of Nonsense Mediated mRNA Decay Associated PI3K Related Kinase (SMG-1). In some embodiments, the compound of Formula III can be referred to as KVS0011.

In some embodiments, two or more of the compounds of Formula I, Formula II, or Formula III, or pharmaceutically acceptable salts thereof can be used to inhibit nonsense mediated decay (NMD). In some embodiments, two or more of the compounds of Formula I, Formula II, or Formula III, or pharmaceutically acceptable salts thereof can be useful as a small molecule inhibitor of Nonsense Mediated mRNA Decay Associated PI3K Related Kinase (SMG-1).

In some embodiments, all three of compounds of Formula I, Formula II, or Formula III, or pharmaceutically acceptable salts thereof can be used to inhibit nonsense mediated decay (NMD). In some embodiments, all three of the compounds of Formula I, Formula II, or Formula III, or pharmaceutically acceptable salts thereof can be useful as a small molecule inhibitor of Nonsense Mediated mRNA Decay Associated PI3K Related Kinase (SMG-1).

### C. Pharmaceutical Compositions

Also provided herein are pharmaceutical compositions that include: (a) one or more of the compound of Formula I, Formula II, or Formula III or pharmaceutically acceptable salts thereof; and (b) a pharmaceutically acceptable carrier. In some embodiments, one or more of the compounds of Formula I, Formula II, or Formula III or pharmaceutically acceptable salts thereof inhibits nonsense mediated decay (NMD). In some embodiments, the cell-surface expression of a neoantigen is increased in a cell following delivering of the pharmaceutical composition into the cell. In some embodiments, the cell-surface expression of the neoantigen is presentation of the neoantigen on a MHC class I HLA molecule. In some embodiments, the neoantigen is derived from a TP53 (tumor protein p53) gene, EXOC1 gene, SPTAN1 gene, RAB14 gene, or ZDHHC16 gene.

In some embodiments, pharmaceutical compositions provided herein are formulated with a pharmaceutically acceptable carrier. As used herein, a “pharmaceutically acceptable carrier” can refer to a substance that aids the administration of an active agent to and absorption by a subject and can be included in the compositions of the present disclosure without causing a significant adverse toxicological effect on the patient. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. In some embodiments, a pharmaceutically acceptable carrier can comprise Kollifor EL (e.g., cremaphor) and glycerol. One of skill in the art will recognize that other pharmaceutically acceptable carriers are useful in the present disclosure.

Pharmaceutical compositions provided herein can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see. e.g., Remington: The Science and Practice of Pharmacy, 21st ed., 2005.

Pharmaceutical composition provided herein can be administered alone or as a component of a pharmaceutical formulation. Pharmaceutical composition provided herein may be formulated for administration, in any convenient way for use in human or veterinary medicine. Pharmaceutical composition provided herein may conveniently be presented in unit dosage form and may be prepared by any methods well known in the art of pharmacy. The amount of active ingredient which can be combined with a carrier material to produce a single dosage form can vary depending upon the host being treated, the particular mode of administration. The amount of active ingredient which can be combined with a carrier material to produce a single dosage form will generally be that amount of the compound which produces a therapeutic effect.

Pharmaceutical compositions described herein can be prepared according to any method known to the art for the manufacture of pharmaceuticals. Such compositions can contain, for example, preserving agents. A composition can be admixtured with nontoxic pharmaceutically acceptable excipients which are suitable for manufacture. Compositions may comprise one or more diluents, emulsifiers, preservatives, buffers, excipients, etc. and may be provided in such forms as liquids, powders, emulsions, lyophilized powders, controlled release formulations, on patches, in implants, etc. Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Aqueous suspensions can contain an active agent (e.g., one or more the compounds of Formula I, Formula II, Formula III, or pharmaceutically acceptable salts thereof) in admixture with excipients suitable for the manufacture of aqueous suspensions, e.g., for aqueous intradermal injections. Such excipients include, without limitation, a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Pharmaceutical compositions can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as egg or soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In some embodiments, these injectable oil-in-water emulsions of the invention comprise a paraffin oil, a sorbitan monooleate, an ethoxylated sorbitan monooleate and/or an ethoxylated sorbitan trioleate.

In some embodiments, pharmaceutical compositions provided herein can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, pharmaceutical compositions provided herein can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed include, without limitation, water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. Formulations provided herein may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in formulations provided herein can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, pharmaceutical compounds and formulations provided herein can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See. e.g., U.S. 20040028670.

Compositions and formulations provided herein can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used herein, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

### D. Methods of Use

Provided herein are methods of inhibiting nonsense mediated decay (NMD), the methods comprising delivering one or more of the compounds of Formula I, Formula II, or Formula III, or pharmaceutically acceptable salts thereof, into a cell, thereby inhibiting NMD in the cell. In some embodiments, provided herein are methods of inhibiting NMD, the method comprising delivering into a cell a compound of Formula I, or a pharmaceutically acceptable salt thereof. In some embodiments, provided herein are methods of inhibiting NMD, the method comprising delivering into a cell a compound of Formula II, or a pharmaceutically acceptable salt thereof. In some embodiments, provided herein are methods of inhibiting NMD, the method comprising delivering into a cell a compound of Formula III, or a pharmaceutically acceptable salt thereof. In some embodiments, one or more of the compounds of Formula I, Formula II, or Formula III, or pharmaceutically acceptable salts thereof are delivered to a cell with a pharmaceutically acceptable carrier. In some embodiments, the cell-surface expression of a neoantigen is increased in the cell. In some embodiments, the neoantigen is derived from a TP53 (tumor protein p53) gene, EXOC1 gene, SPTAN1 gene, RAB14 gene, or ZDHHC16 gene.

Also provided herein are methods of enhancing immunotherapy in a subject in need thereof, the method comprising administering to the subject one or more of the compounds, or pharmaceutical compositions described herein.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In some embodiments, compounds or pharmaceutical compositions provided herein inhibit nonsense mediated decay (NMD). In some embodiments, cell-surface expression of a neoantigen is increased in the subject following administration of the compound or the pharmaceutical composition. In some embodiments, the neoantigen is derived from a TP53 (tumor protein p53) gene, EXOC1 gene, SPTAN1 gene, RAB14 gene, or ZDHHC16 gene. In some embodiments, the subject has been identified or diagnosed as having a cancer.

### E. Methods of Treatment

Also provided herein are methods of treating cancer in a subject in need thereof, the method comprising administering to the subject: (a) one or more of the compounds of Formula I. Formula IL or Formula III, or pharmaceutically acceptable salts thereof; and (b) an immunotherapy. In some embodiments, provided herein are methods of treating cancer in a subject in need thereof, the method comprising administering to the subject: (a) a compound of Formula I, or a pharmaceutically acceptable salt thereof; and (b) an immunotherapy. In some embodiments, provided herein are methods of treating cancer in a subject in need thereof, the method comprising administering to the subject: (a) a compound of Formula II, or a pharmaceutically acceptable salt thereof; and (b) an immunotherapy. In some embodiments, provided herein are methods of treating cancer in a subject in need thereof, the method comprising administering to the subject: (a) a compound of Formula III, or a pharmaceutically acceptable salt thereof; and (b) an immunotherapy. In some embodiments, one or more of the compounds of Formula I, Formula II, or Formula III, or pharmaceutically acceptable salts thereof are administered to a subject with a pharmaceutically acceptable carrier.

As used herein, “immunotherapy” refers to a treatment of disease (e.g., cancer) by activating or suppressing the immune system. For example, cancer immunotherapy uses the immune system and its components to mount an anti-tumor response through immune activation. In some embodiments, an immunotherapy can include an immune checkpoint inhibitor, an oncolytic virus therapy, a cell-based therapy, a CAR-T cell therapy, or a cancer vaccine. In some embodiments, an immunotherapy can include immune checkpoint blockade, wherein an immune checkpoint inhibitor is administered. In some embodiments, the immune checkpoint inhibitor can be any checkpoint inhibitor, e.g., as described in Mazzarella et al., Eur J Cancer (2019) 117:14-31, hereby incorporated by reference.

In some embodiments, the method further comprises administering to the subject one or more additional anticancer therapies. In some embodiments, the one or more additional anticancer therapies comprise a chemotherapeutic agent, ionizing radiation, a therapeutic antibody, or gene therapy. As used herein, an “anticancer agent” or “anticancer therapy” refers to a molecule (e.g. compound, peptide, protein, nucleic acid) or treatment used to treat cancer through destruction or inhibition of cancer cells or tissues. Anticancer therapies may be selective for certain cancers or certain tissues. In embodiments, anticancer therapies herein may include epigenetic inhibitors and multi-kinase inhibitors.

In some embodiments, compounds or pharmaceutical compositions provided herein inhibit nonsense mediated decay (NMD). In some embodiments, the cell-surface expression of a neoantigen is increased in the subject following administration. In some embodiments, the neoantigen is derived from a TP53 (tumor protein p53) gene, EXOC1 gene, SPTAN1 gene, RAB14 gene, or ZDHHC16 gene.

In some embodiments, the cancer is a solid tumor, hematological tumor, sarcoma, osteosarcoma, glioblastoma, neuroblastoma, melanoma, rhabdomyosarcoma. Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic mveloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

### EXAMPLES

### Example 1—Development of a High Throughput Assay to Identify NMD Inhibitors

An experimental system was generated to identify and evaluate NMD inhibitors based on a panel of isogenic cell lines developed to assess tumor suppressor gene function and synthetic lethality. Briefly, common tumor suppressor genes were individually knocked out in multiple non-cancerous cell lines using the Clustered Regularly Interspaced Short Palindromic Repeats (CRISPR)-Cas9 system. As part of this effort, two genes displaying strong activation of NMD in response to indel creation were identified, Stromal Antigen 2 (STAG2) and Tumor Protein p53 (TP53). The absence of detectable protein in these knock out cell lines was verified via western blots against STAG2 and immunohistochemistry for TP53. Further, active RNA decay of the mutant transcripts was identified through whole transcriptome RNA-sequencing. An average decrease of STAG2 RNA transcripts by 20-fold and TP53 by 6-fold relative was shown to their respective wild type transcripts in the parent cell line.

Two STAG2 and one TP53 isogenic knockout cell lines were selected from the RPE1 background to design a high-throughput screen (HTS) based on their engineered mutation eliciting a strong NMD response. A unique sequencing assay was designed built on two primer sets amplifying the common mutant region of STAG2 and mutant region of TP53. Plating out cell line mixed in equal proportions it was possible to simultaneously query the mutant and wild type transcripts, comparing their expression levels. Using this ratio metric assay, a 6-fold recovery of mutant RNA transcript expression upon treatment with canonocial NMD inhibitor, emetine, was demonstrated. Human clinical trials suggest compound specific toxicity at doses required for NMD inhibition makes it and other previously identified inhibitors unsuitable for human NMD targeted therapy.

### Example 2—High Throughput Screen to Identify NMD Inhibitors

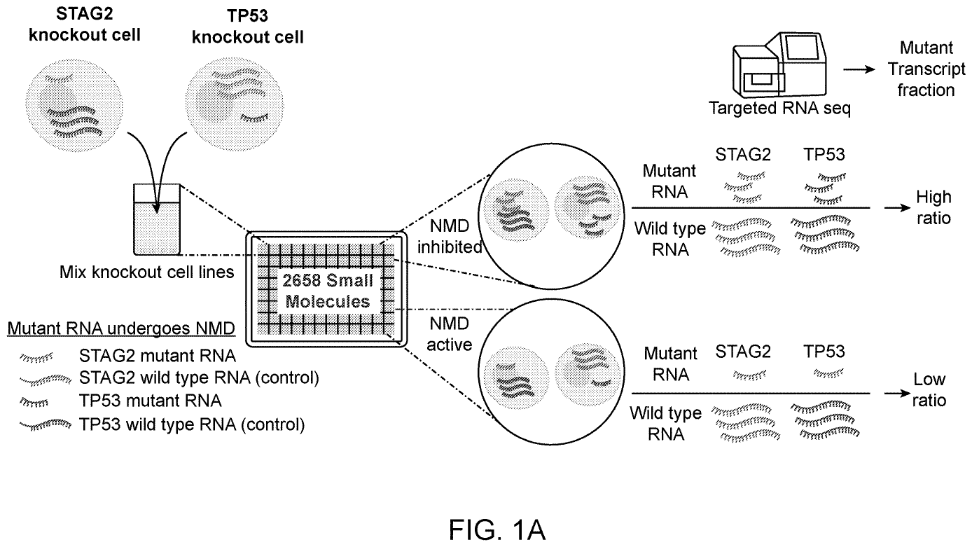

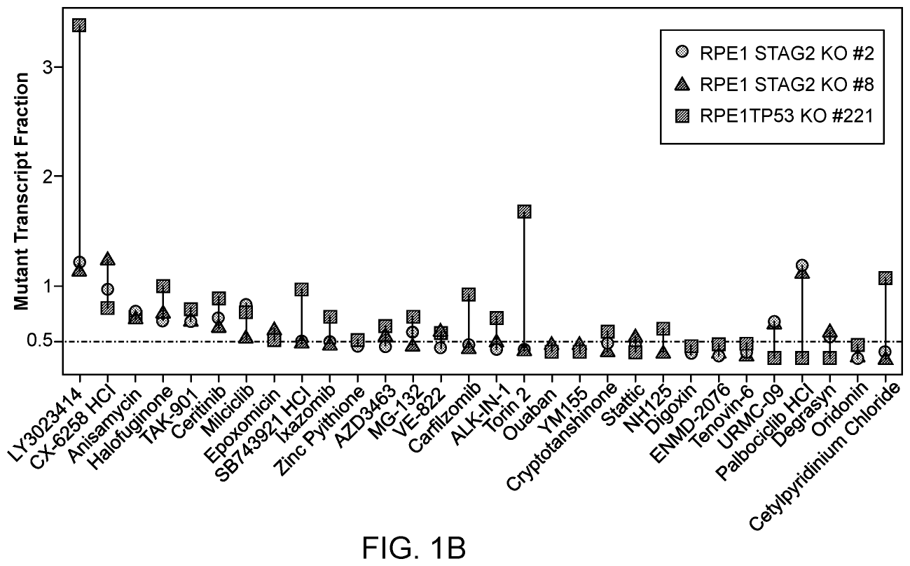

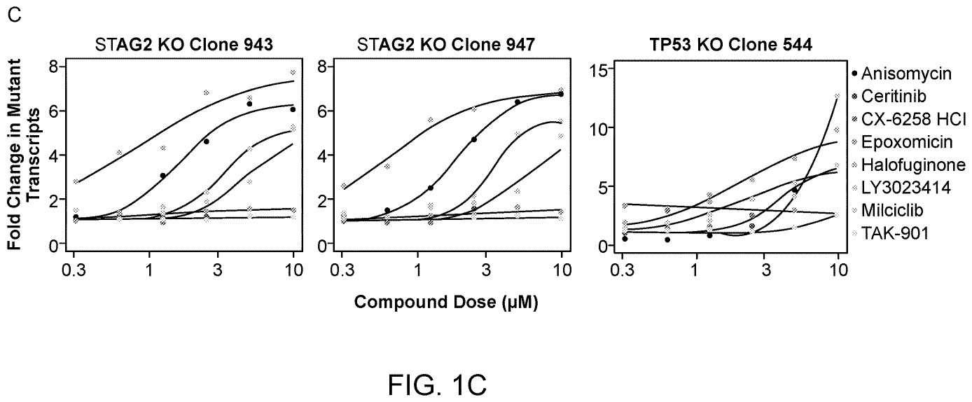

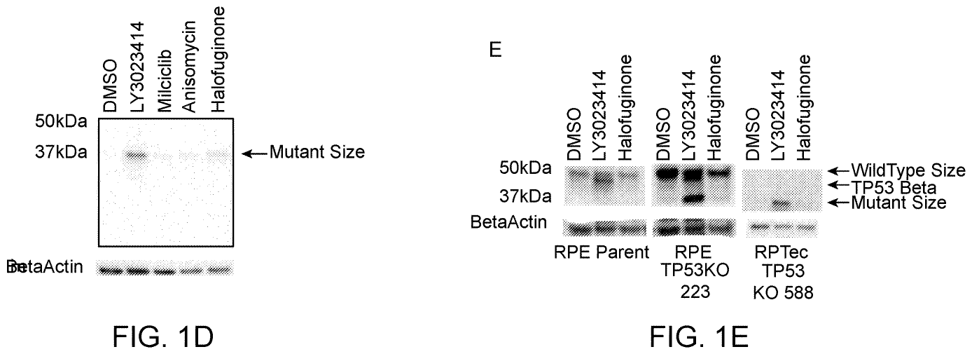

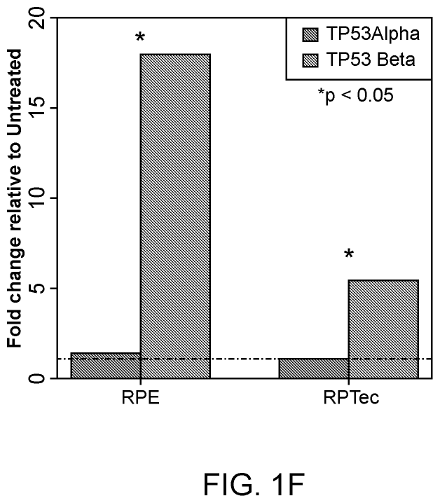

A high throughput screen (HTS) was then performed by treating the isogenic cell line mixture with the SelleckChem Bioactive library, consisting of 2,658 small molecules and natural products (FIG. 1A). Total RNA was harvested from cells 16 hours post treatment and the gRNA target regions were amplified using a panel of primers (one for TP53 and one for STAG2) and mutant and wild type transcript abundance was quantified using next generation sequencing. The wild type sequence of the reciprocal knocked out clone, e.g. STAG2 sequence from the TP53 knockout clones, and TP53 sequence from the STAG2 knockout clones, served as an internal reference to create a ratiometric assay of mutant to wild type transcript abundance (FIG. 1A). Using a ratio as the result eliminated bias introduced from nonspecific transcriptional activators, or compounds which were universally toxic. Only eight compounds (0.3% of the library) increased mutant transcript fraction above 0.5 (4 standard deviations above the DMSO controls) in all three cell lines (FIG. 1B). One of these eight compounds was Anisomycin, an inhibitor of protein synthesis and NMD inhibitor, providing independent validation of screen. Overall the screen identified compounds which increased mutant RNA transcription on average between 5 and 10 fold relative to untreated cells.

### Example 3—LY3023414 Inhibits NMD and Cause Re-Expression of Mutant RNA and Protein

To confirm generality of the NMD HTS hits, validations were performed on isogenic STAG2 and TP53 knockouts in a second cell line background, RPtec (renal) cells. Transcript recovery was first evaluated in STAG2 and TP53 isogenic knockout clones from this second background. Of the eight hit compounds, it was found that four increase mutant RNA expression in a dose responsive manner in all three isogenic clones as measured by targeted RNA-sequencing (FIG. 1C). A TP53 knockout cell line was then selected with a homozygous mutation predicted to generate a truncated TP53 protein readily identifiable by established TP53 antibodies (DO-1). This specific cell line was evaluated against the four reproducible NMD inhibitors and found LY3023414 and halofuginone robustly induced expression of the truncated mutant TP53 protein (FIG. 1D). Interestingly, TP53 has an isoform TP5³10 which is known to downregulated by the NMD pathway. Using cell lines where the TP530 isoform transcripts are expected to be intact, an increase in the TP53$ isoform was also observed in cell lines treated with the NMD inhibitors (FIG. 1E middle and left column). Further analysis by quantitative real time PCR evaluation of both the full length TP53 (aTP53α) and NMD sensitive transcripts (TP53β) in parental RPE1 and RPTec cells treated with LY3023414 confirmed only the NMD regulated (TP53β) transcript levels increased with treatment (FIG. 1F). Based on strong induction of predicted mutant truncated protein across multiple isogenic cell lines LY3023414 was selected as the NMD inhibiting compound of interest. LY3023414 was created as a phosphatidylinositol 3-kinase (PI3K) inhibitor with activity against AKT Serine/Threonine Kinase 1 (AKT1) and Target of Rapamycin (mTOR).

### Example 4—LY3023414 Increases Expression of Many Indel Containing RNA Transcripts and Mutant Proteins In-Vitro and In-Vivo

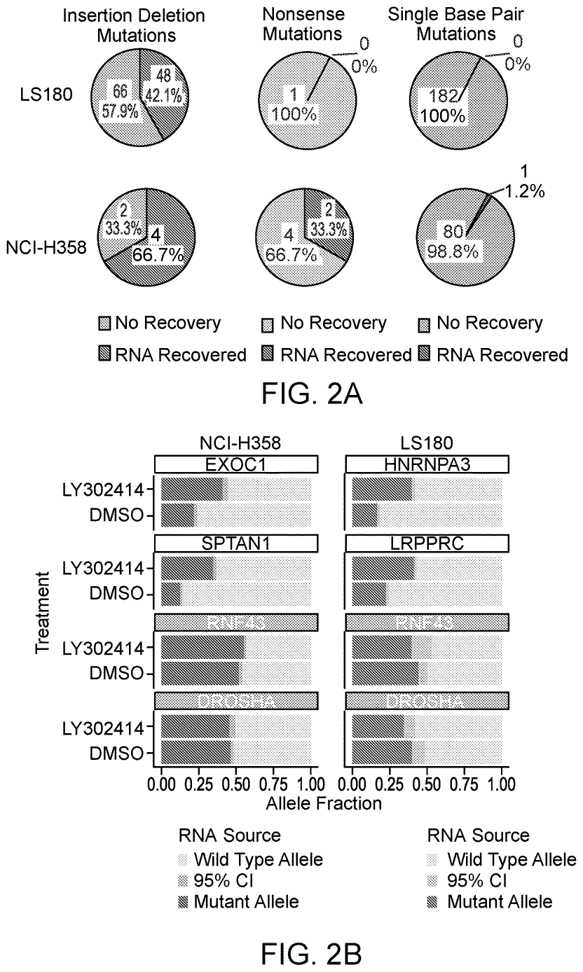

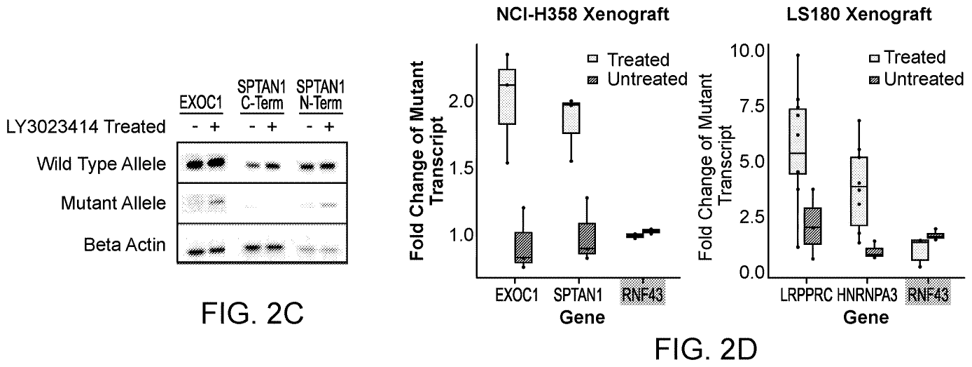

It was next sought to evaluate whether LY3023414 could relieve repression of indel containing transcripts in cancer cells. The microsatellite stable NCI-H358 and microsatellite instable LS180 lung and colorectal cancer cell lines were chosen respectively. Nearly 40% of heterozygous indel and 25% of nonsense single base-pair mutation containing genes show increased expression of their mutant allele when treated with LY3023414 when evaluated by RNA sequencing (FIG. 2A). Targeted RNA sequencing of two heterozygous indel mutations from each cell line confirmed significant increased expression of the mutant allele when treated with LY3023414 (FIG. 2B). Heterozygous single nucleotide polymorphism (SNP) Ring Finger Protein 43 (RNF43) and drosha ribonuclease 111 (DROSHA) served as controls, and allele ratios remain unchanged with treatment (FIG. 2B). To investigate for changes at the protein level, NCI-H358 cells were treated for 24 hours with either LY3023414 or DMSO. Western blot revealed the full-length proteins, Exocyst Complex Component 1 (EXOC1) and Spectrin Alpha, Non-Ervthrocytic 1 (SPTAN1), was present in all samples, with the truncated mutant protein only expressed after treatment with LY3023414 (FIG. 2C). An antibody for SPTAN1 specific to residues downstream (C-terminal) of the frameshift failed to detect mutant protein as expected (FIG. 2C middle).

LY3023414 was developed and tested in a number of clinical trials and has a well-known pharmacokinetic profile for in-vivo experiments. To evaluate whether it disrupts NMD in-vivo, xenograft tumors of both NCI-H358 and LS180 were established in NOD-SCID (NOD.CB17-Prkdcscid/NCrCrl) mice. These mice were then treated with a single dose of LY3023414 by oral gavage and harvested tumors over 24 hours. An increase in the expression of mutant RNA transcripts was observed in the tumors (FIG. 2D). However, severe weight loss limited observation of the long-term effects of LY3023414 therapy on tumor growth. Autopsies of treated animals revealed no abnormalities and suggests additional toxic effects in immune competent mice.

### Example 5—the Kinase SMG1 is the Target for NMD Inhibition by LY3023414

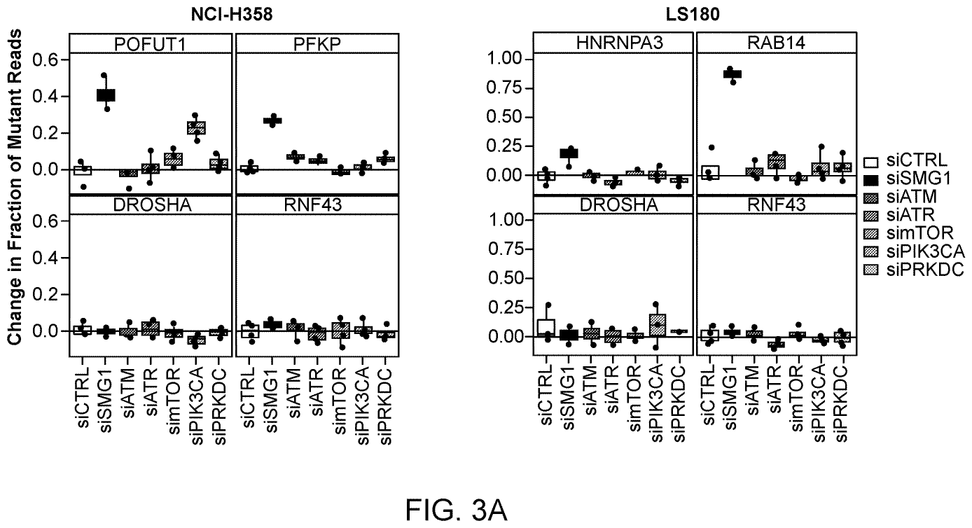

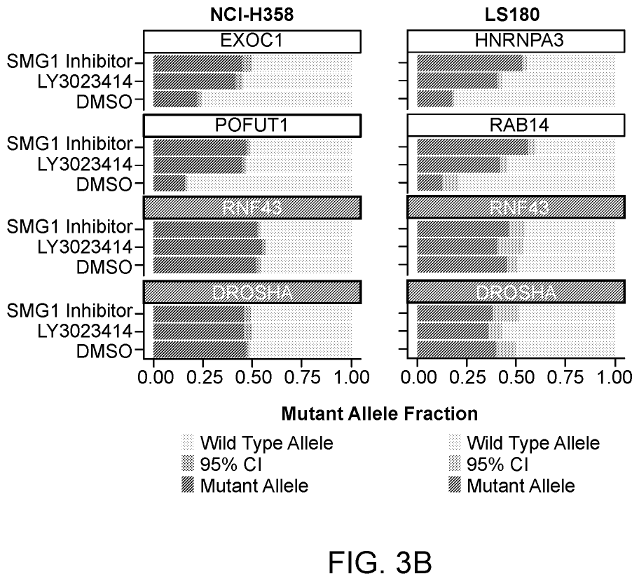

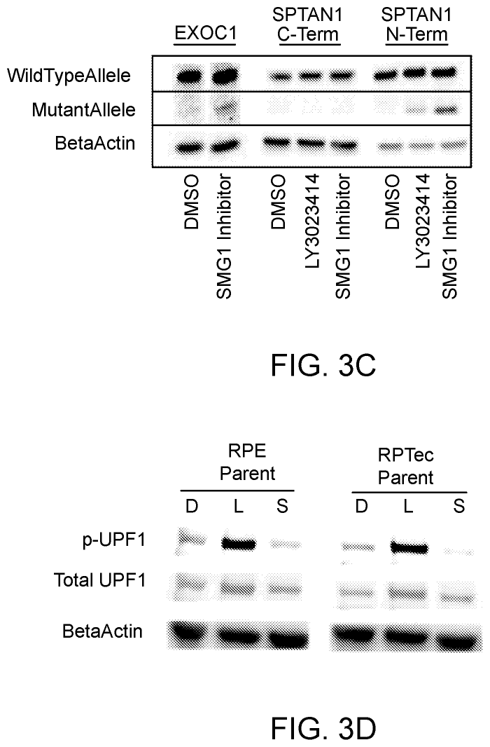

Given that LY3023414 has multiple potential targets and dose limiting toxicity for NMD inhibition, the target of its NMD inhibition was searched for. It was indicated that this compound is a broad kinase inhibitor, so the six kinases with the highest reported inhibition by LY3023414 were selected and siRNA knockdown of each were performed in both NCI-H358 and LS180 cancer cell lines. Only knockdown of Nonsense Mediated mRNA Decay Associated PI3K Related Kinase (SMG1) caused mutant transcript expression in every evaluated gene (FIG. 3A). Because siRNA are known to have off-target effects, it was sought to further evaluate the role of SMG inhibition in NMD suppression. SMG1 is a kinase which functions in the NMD pathway by activating the UPF1 RNA helicase and ATPase (UPF1). Inhibition of UPF1 activation by siRNA knockdown of UPF1 induced expression of NMD downregulated transcripts to a similar extent as SMG knock down further supporting the role of the of SMG pathway inhibition as the target NMD suppression.

To orthogonally confirm the siRNA results, a previously described small molecule was obtained with picomolar affinity and specificity greater than 400-fold for SMG1 over other similar kinases (e.g. mTOR). Treatment of NCI-H358 and LS180 cancer cell lines with this SMG1 specific inhibitor showed mutant specific expression of both RNA and protein in both cell lines (FIGS. 3B-3C). Analyzing a wider set of 21 NMD sensitive genes from the RNA-Seq analysis in LS180 confirmed that LY3023414 and SMG1 specific inhibitor increase the same 18 genes with a greater than 80% re-expression rate, in both cell lines. To confirm on target activity, a phopho-UPF1 specific western was performed and, surprisingly, it was found that LY3023414 increases the levels of phospho-UPF, while the SMG1 specific inhibitor causes an expected decreased phosphorylation (FIG. 3D). As SMG1 inhibition is an off target effect for LY3023414, the concentrations used in this study may result in inhibition of a number of different kinases. Previous reports show that when under significant stress, such as growth in the presence of a pan-kinase inhibitor, cells will hyper-phosphorylate UPF1 in an attempt to remove mutant or unneeded transcripts.

### Example 6—Development of Improved NMD Inhibitors Targeting SMG1

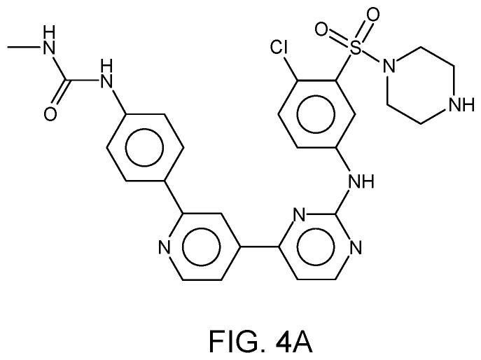

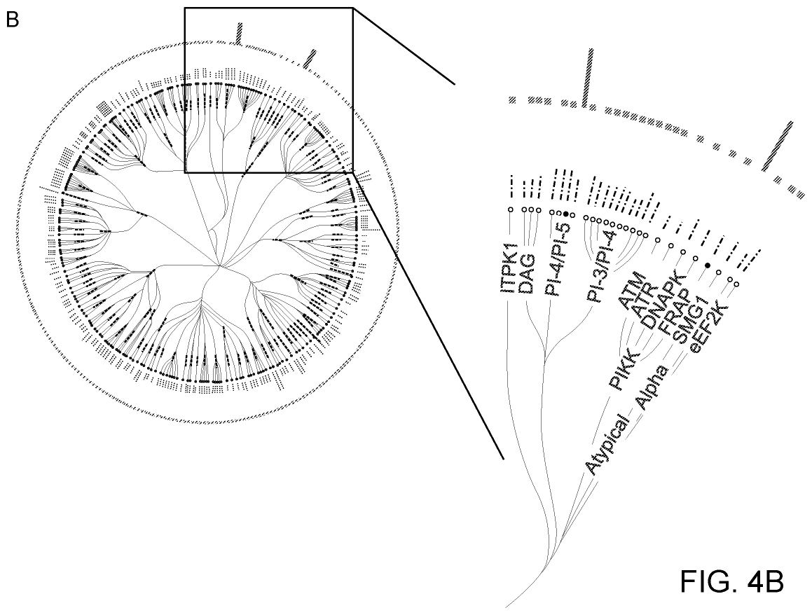

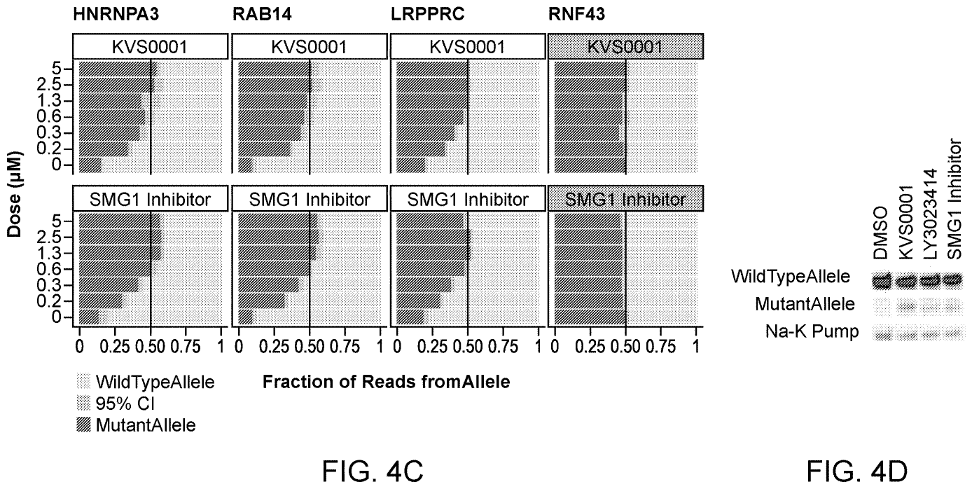

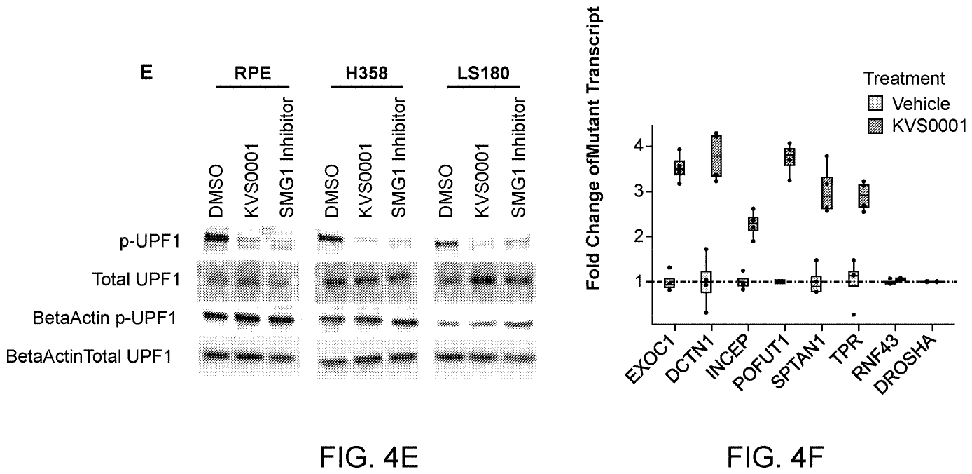

To overcome the off target toxicity of LY3023414 and solubility issues of previously made SMG1 specific small molecule inhibitors, a derivative of the SMG inhibitor (the compound of Formula I) was synthesized that preserved the ability to inhibit SMG while displaying more desirable physical properties related to solubility (FIG. 4A). Kinativ™ assay performed on this novel inhibitor confirms it maintains high specificity for SMG1 protein over other kinases (FIG. 4B). Dose response curves on NCI-H358 and LS180 cells treated in-vitro confirmed that the compound of Formula I is bioactive in the nanomolar range, similar to the previously described SMG1 inhibitor (FIG. 4C). Treatment with the compound of Formula I was also found to induce expression of the NMD targeted mutant protein from NCI-H358 and LS180 cells (FIG. 4D). To confirm disruption of downstream signaling of SMG1, a western blot was performed with phosphorylation specific antibodies for UPF1. Treatment with the compound of Formula I strongly decreased the amount of phosphorylated UPF1 in three different cell lines, confirming its activity regardless of tissue background (FIG. 4E). Xenografts in NOD-SCID mice containing NCI-H358 and LS180 subcutaneous tumors treated intraperitoneal (IP) with the compound of Formula I display increases in mutant transcript levels relative to untreated mice, confirming in-vivo activity of our novel compound (FIG. 4F). Mice did not show any toxicity related behavior (e.g., hunching, lethargy) with doses up to 50 mg/kg.

### Example 7—NMD Inhibition Causes MHC Class I Display of Hidden Neoantigens

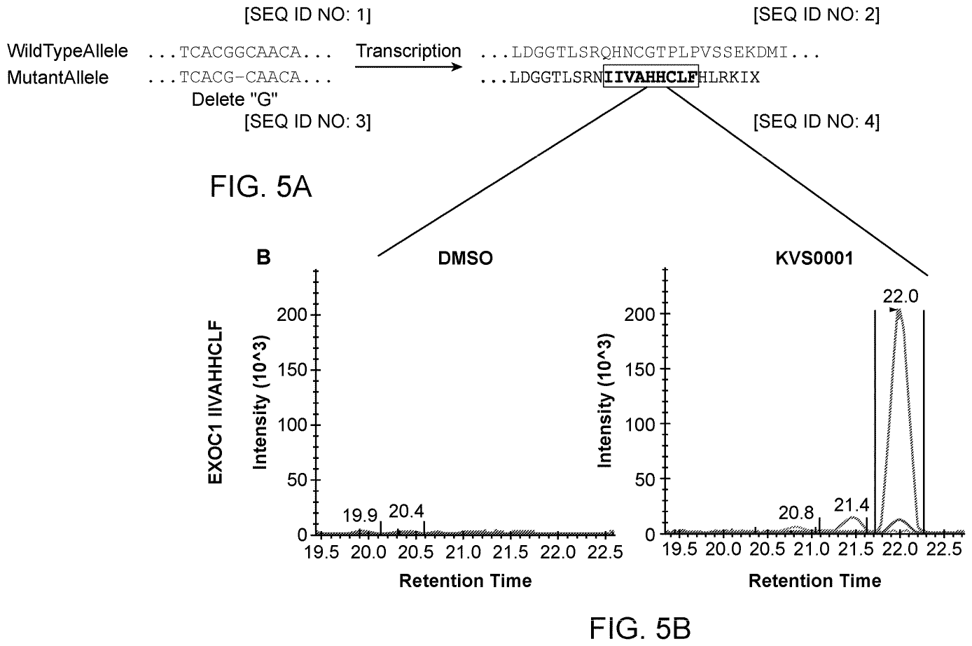

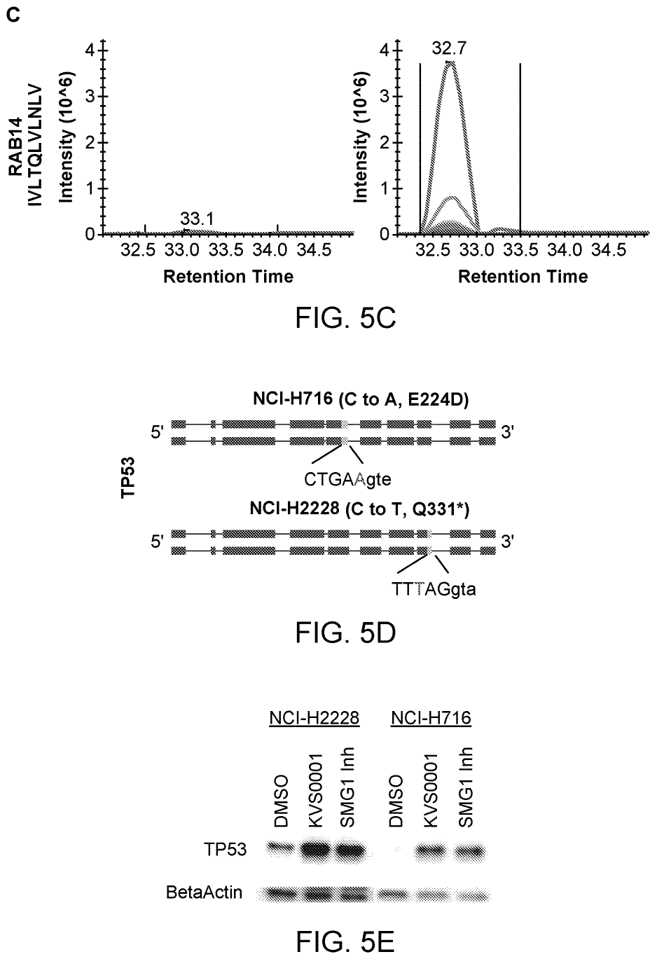

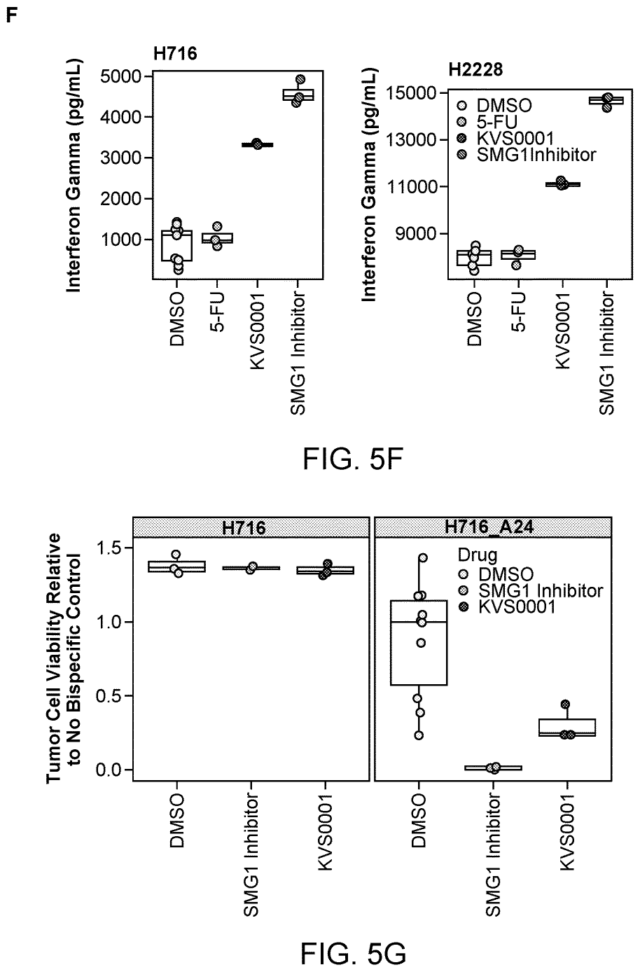

Downregulating mutated genes with frame shifts via NMD is one mechanism employed by cancer cells to avoid immune surveillance. To confirm the small molecule could alter the immune landscape of a tumor, cell surface presentation of known mutant proteins was assessed in NCI-H358 and LS180 cells. Cells were treated in-vitro for 24 hours with DMSO or the compound of Formula I in duplicate, cross linked and immunoprecipitated with pan-HLA antibodies to obtain cell surface MHC presented peptides. 187 mutation specific peptides were initially identified that could be potentially presented by using the union of NetMHC and Predictor of Immunogenic Epitopes (PRIME) predictions. These predictions were tested by performing HPLC Mass Spectromety (MS) on cells that were treated in-vitro for 24 hours with DMSO or the compound of Formula I, cross linked and immunoprecipitated with pan-HLA antibodies to obtain cell surface MHC presented peptides. Based on those results, heavy isotope labeled peptides were designed for 14 peptides from NCI-H358, and 24 peptides from LS180 cells with high confidence of MS detection. These heavy labeled peptides were then used as internal standard to perform quantitative HPLC-Mass Spectrometry. This analysis revealed the presence of a neoantigen specific to the mutant alleles of two genes in NCI-H358 and two other genes in LS180 cells treated with the compound of Formula I (FIGS. 5A-5B). This is consistent with re-expression of mutant protein shown previously by western blot (FIGS. 3C and 4D). These observations support extant data showing cell surface presentation of novel antigens from tumor specific mutations when NMD is pharmacologically inhibited. These previous studies repurpose existing drugs to inhibit NMD though and risk off target effects, unlike the compound of Formula I which is designed only to inhibit SMG1.

### Example 8—Targetable Peptide Presentation Occurs with NMD Inhibition

To test whether a neoantigen peptide presented only when NMD is inhibited could be targeted two model cell lines were chosen: NCI-H716 and NCI-H2228. Both contain homozygous mutations in the TP53 gene, with NCI-H716 occurring immediately adjacent to exon-intron boundary and NCI-2228 introducing an early stop; two mutation types which commonly instigate an NMD response (FIG. 5D). Publicly available data confirm both of these cell lines exhibit very low RNA transcript levels for TP53. Treatment with nonspecific DNA damage agents, 5-fluorouracil and etoposide, had no effect on TP53 levels, whereas treatment with NMD inhibitors displayed increased expression of the mutant form of TP53 protein in both cell lines (FIG. 5E).

To determine whether this relief of repression is targetable, a bispecific antibody targeting wild type TP53 and CD3 was employed. Treatment of NCI-H716 and NCI-2228 with bispecific antibody, human CD8+ T-cells and the compound of Formula I caused an increase in interferon gamma release based on an ELISA assay (FIG. 5F). Treatment without bispecific antibody led to no change in interferon gamma levels. To assess if these novel antigens could be utilized for immune driven cell specific killing, NCI-H716 and NCI-2228 cell lines with and without HLA-A24 expression were labeled with luciferase. Treatment with the compound of Formula I, human CD8+ T-cells, and bispecific antibody demonstrated cell killing only in cells with HLA-A24 present (FIG. 5G). To establish this effect remained in-vivo, NCI-H716 cells expressing HLA-A24 and luciferase were placed orthotopically in NSG mice. Tumor size was measured by luciferase readings via bioluminescence imaging (BLI). Combination therapy significantly decreased the luminescence signal relative to vehicle, or bispecific alone.

### Example 9—Tumor Growth is Slowed in Immune Competent Mouse Models Treated with the Compound of Formula I

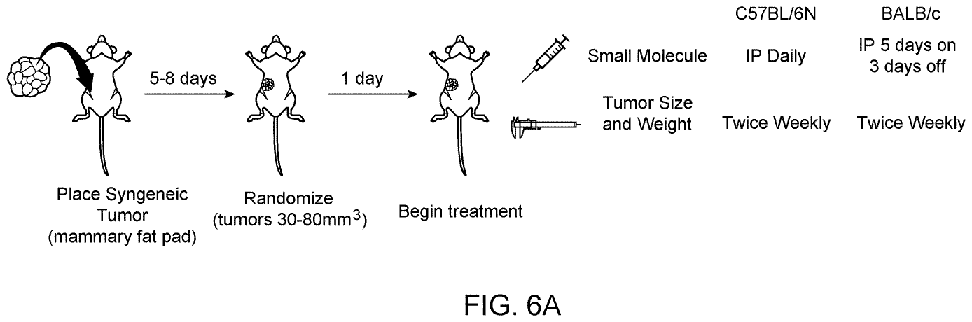

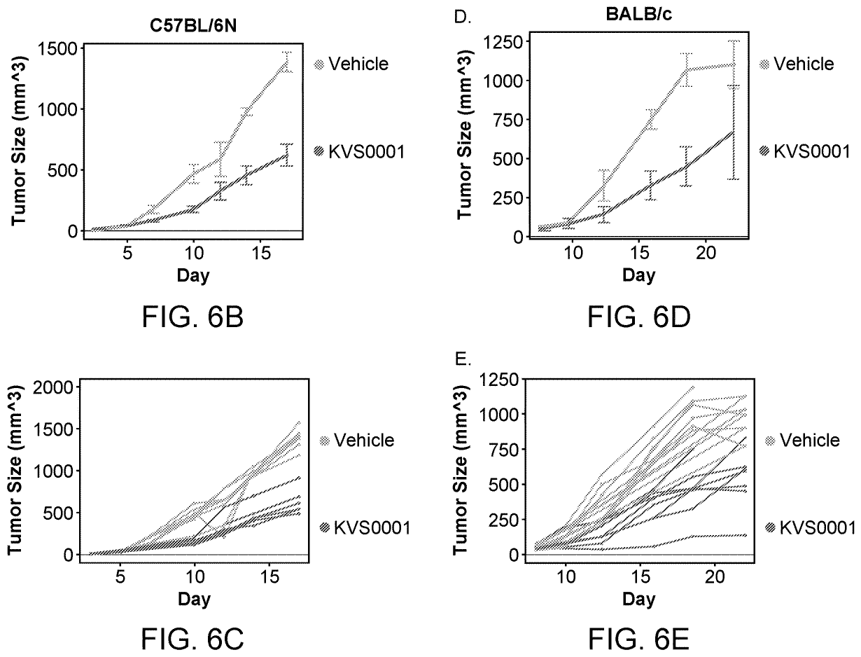

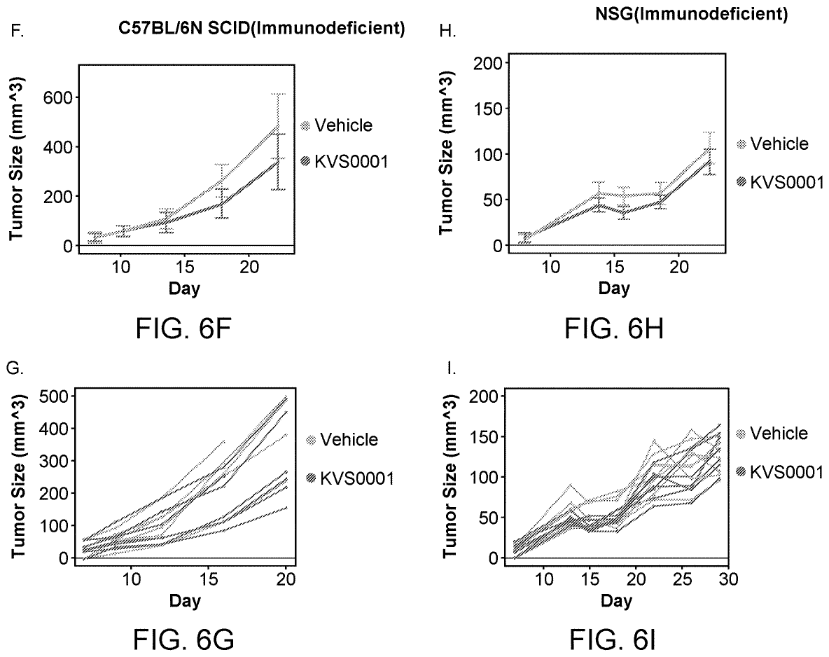

Confident NMD inhibition with the compound of Formula I was leading to in-vivo changes to the tumor and its immune environment and it was evaluated whether these alterations could be taken advantage of in an immune competent system. Murine RENCA (renal) and LLC (Lewis Lung Cancer) cell lines were chosen as they harbor a large number of out of frame indel mutations based on whole exome DNA sequencing (WES) (Table SX-RENCA and LLC mutations). It was confirmed that treatment of these cell lines could stop NMD mediated repression of mutant RNA, and induced expression of exogenous genes controlled by NMD by real time PCR. Cells were placed in the mammary fat pad of C57BL/6N (LLC also known as LL2) and BALB/c (RENCA) mice, randomly sorted animals after tumors became palpable ensuring to balance the tumor size of each group, and then treated with vehicle or the compound of Formula I (FIG. 6A). Significant slowing of tumor growth was observed in both models (FIGS. 6B-6E). The same experiment repeated in immunosuppressed mice showed minimal or no difference in cell growth (FIGS. 6F-6I). Some weight loss was noted in the compound of Formula I arm in the BALB/c background, but was within tolerable limits. No weight loss was appreciated in the C57BL/6N background. This experiment was also attempted in a number of other syngeneic lines but only the melanoma line B16-F10 showed a change in tumor volume with the compound of Formula I treatment.

### Example 10—Compound of Formula II RNA Re-Expression

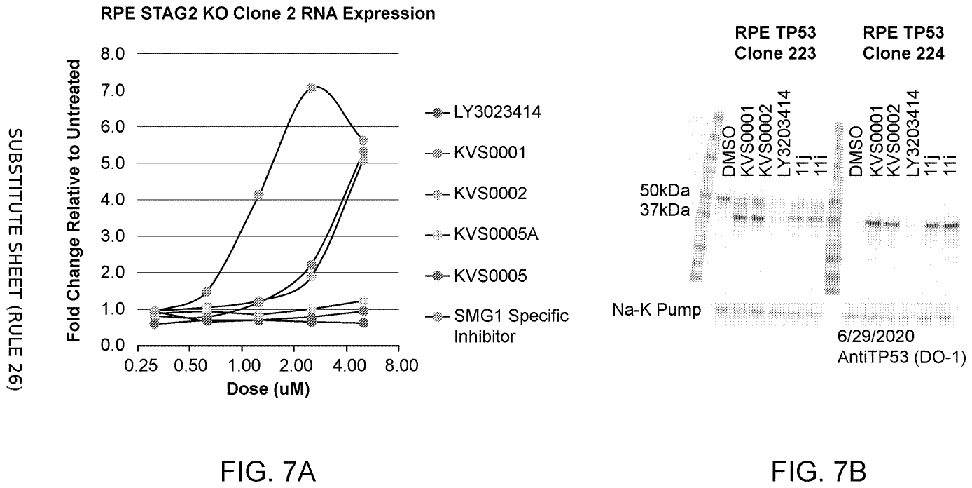

RPE STAG2 knockout cells were created and plated into 96 well plates at 1e4 cells/well. The compound of Formula II was solubilized in DMSO and added in a serial dilution starting at 10 uM and diluting 1:2. Cells were incubated for 16 hrs at 37 C and 5% CO2. RNA was harvested using an RNeasy kit (Qiagen). Real time PCR was performed using primers specific for STAG2 transcripts and at least one primer overlapped an exon-exon boundary (FIG. 7A).

### Example 11—Compound of Formula II Protein Re-Expression

TP53 KO lines were created and plated (at 1e5 cells per well) into 6 well plates. Wells were treated with 5 uM of the compound of Formula II for 16 hours. Cells were harvested, pelleted and washed with PBS. Cells were lysed using RIPA buffer (Thermo Scientific) and then run through a Qiashredder column (Qiagen) following manufacturer's instructions. Protein was quantified using a BCA Kit (Pierce). 50 ug of protein was loaded into 5-15% polyacrylamide gels (Biorad) and run for 30 min at 200V. Protein was transferred to a nitrocellulose membrane following manufacturer's recommendations and stained with a TP53 protein (clone DO-1 from Cell Signaling) overnight in 3% milk with TBS-T. HRP conjugated secondary antibody was used for detection and imaged on a gel imager (Bio Rad) (FIG. 7B).

### Example 12—Compound of Formula III RNA Re-Expression

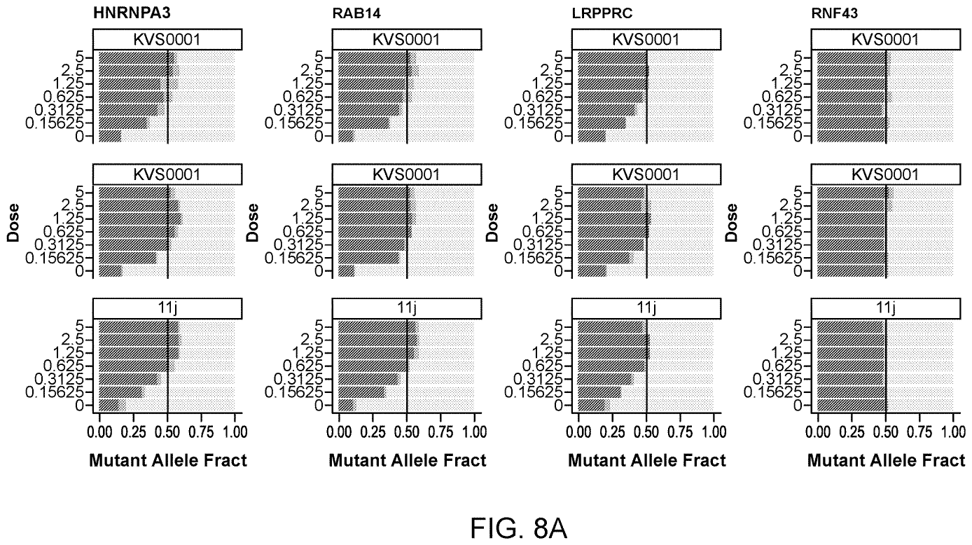

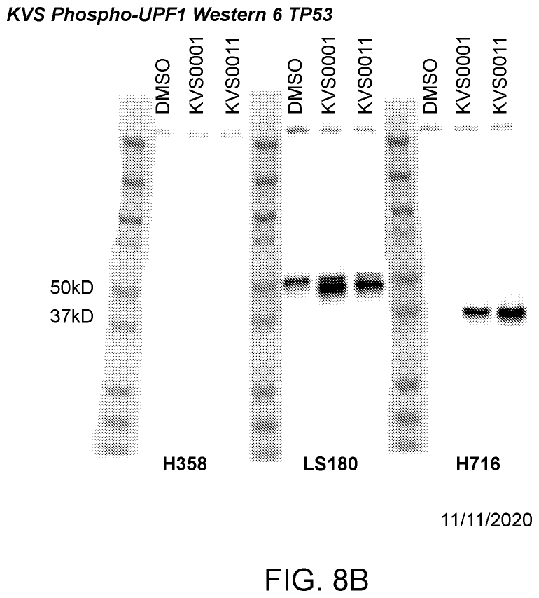

LS180 Cells were plated into 96 well plates at 1e4 cells/well. The compound of Formula III was solubilized in DMSO and added in a serial dilution starting at 5 uM and diluting 1:2. Cells were incubated for 16 hrs at 37 C and 5% CO2. RNA was harvested using an RNeasy kit (Qiagen). Reverse transcription was performed per manufacturer's instructions (High Capacity cDNA kit from Applied Biosciences). Primers specific for the indel mutation site for 3 genes were used, and at least one primer from each set overlapped an exon-exon boundary. RNF43 is a common SNP that is heterozygous in LS180 and was used as a negative control. After PCR amplification, products were barcoded with an 8 bp sequence so the well it originated in could be determined, and sequenced on a Miseq (Illumina). Transcripts from the wild type and mutant allele were counted to determine abundance of each with treatment or without (FIG. 8A).

### Example 13—Compound of Formula III Protein Re-Expression

H358 (negative control), LSl80, and RPE TP53 KO line clone 224 were plated (at 1e5 cells per well) into 6 well plates. Wells were treated with 5 uM of the compound of Formula III for 16 hours. Cells were harvested, pelleted and washed with PBS. Cells were lysed using RIPA buffer (Thermo Scientific) and then run through a Qiashredder column (Qiagen) following manufacturer's instructions. Protein was quantified using a BCA Kit (Pierce). 50 ug of protein was loaded into 5-15% polyacrylamide gels (Biorad) and run for 30 min at 200V. Protein was transferred to a nitrocellulose membrane following manufacturer's recommendations and stained with a TP53 protein (clone DO-1 from Cell Signaling) overnight in 3% milk with TBS-T. HRP conjugated secondary antibody was used for detection and imaged on a gel imager (Bio Rad) (FIG. 8B).

### Example 14—Compound of Formula III In Vivo Data

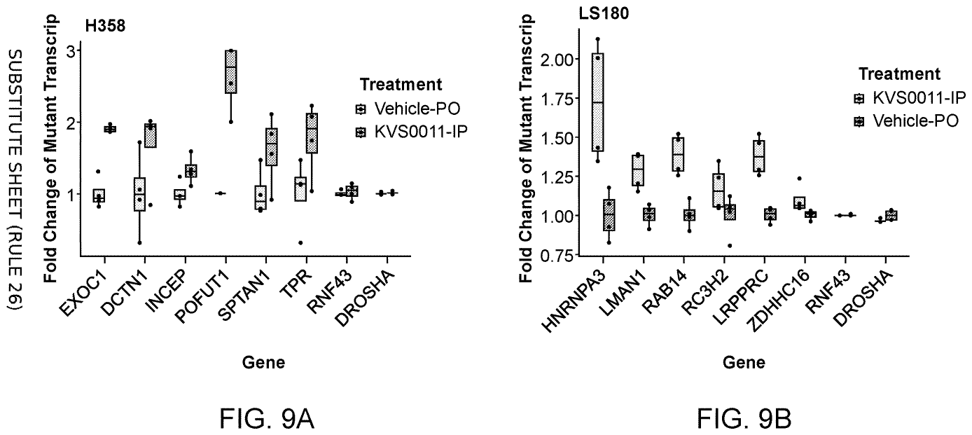

All animal work was done under the supervision and guidance of Johns Hopkins Animal Care and Use committee with appropriate protocols and humane standards of care. LS180 and H358 cell lines (ATCC) were placed subcutaneously in the back of nude mice (Jackson Laboratories). The compound of Formula III was solubilized in 10% cremaphor (Sigma Aldrich) with 2% glycerol (Sigma Aldrich) and sterilized by 0.22 uM filter (Thermo Scientific). After tumors reached palpable size, about 7 days, mice were treated with 30 mg/mL of the compound of Formula III by IP injection. 16 hours after treatment, mice were sacked, and tumors were harvested and stored in RNA Later (Thermo Scientific). RNA was harvested using an RNeasy kit (Qiagen) and Qiashredder (Qiagen). Reverse transcription was performed per manufacturer's instructions (High Capacity cDNA kit from Applied Biosciences). Primers specific for the indel mutation site for 3 genes were used, and at least one primer from each set overlapped an exon-exon boundary. RNF43 and DROSHA are common SNP's that are heterozygous in LS180 and H358 cell lines and were used as a negative control. After PCR amplification, products were barcoded with an 8 bp sequence so the well it originated in could be determined, and sequenced on a Miseq (Illumina). Transcripts from the wild type and mutant allele were counted to determine abundance of each with treatment or without (FIGS. 9A-9B).

### Example 15—Compounds of Formula (V)

Exemplary compounds of Formula (V) and associated data are provided in Tables 1 and 2, respectively, below.

TABLE 1

| Compound | |
|---|---|
| Number | Structure |
| 1 | 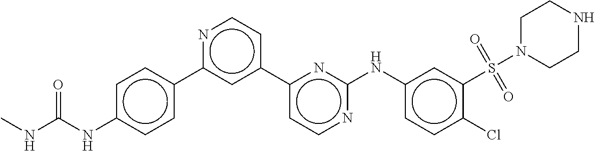 |
| 2 | 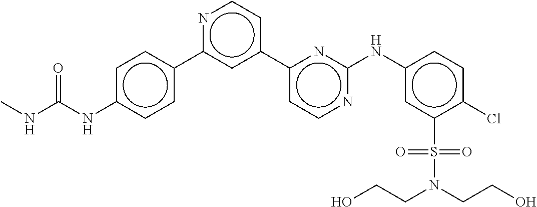 |
| 3 | 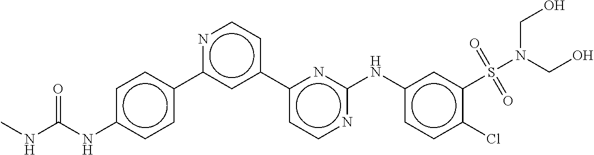 |
| 4 | 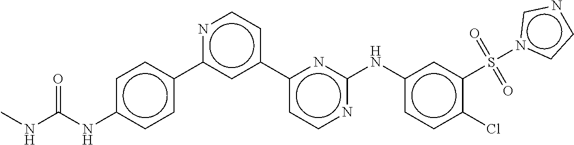 |
| 5 | 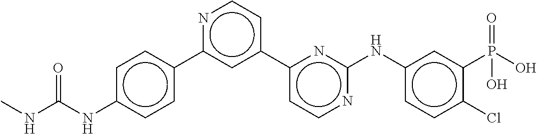 |
| 6 | 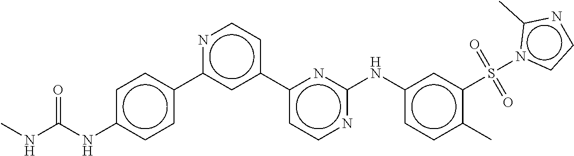 |
| 7 | 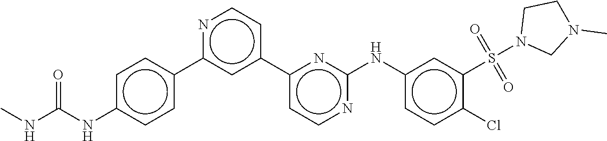 |
| 8 | 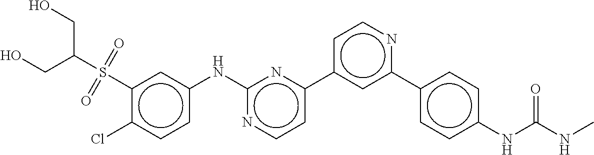 |
| 9 | 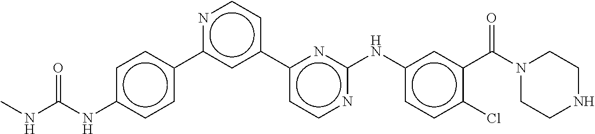 |
| 10 | 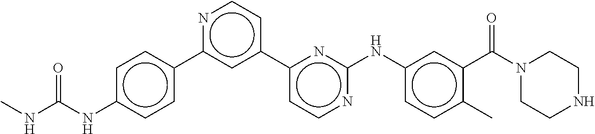 |
| 11 | 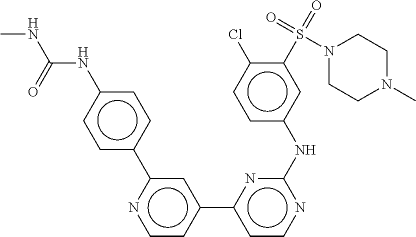 |
| 12 | 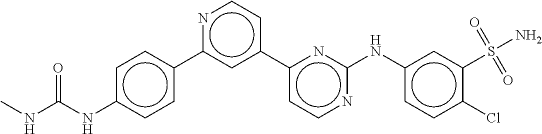 |
| 13 | 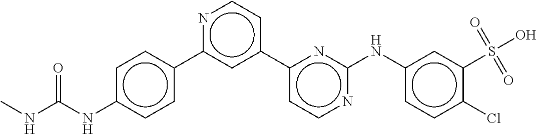 |
| 14 | 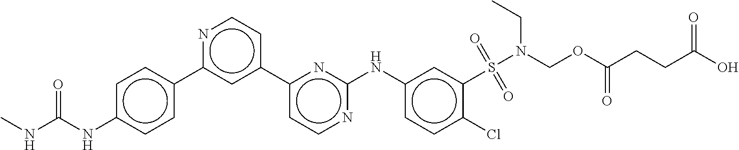 |
| 15 | 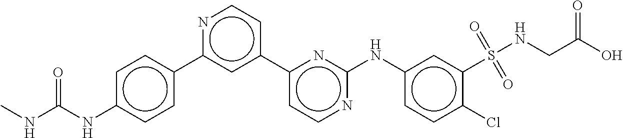 |
| 16 | 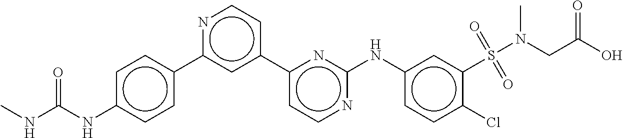 |
| 17 | 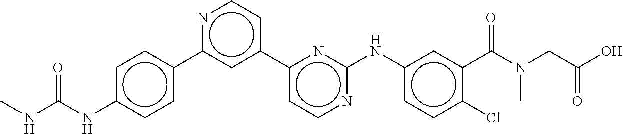 |
| 18 | 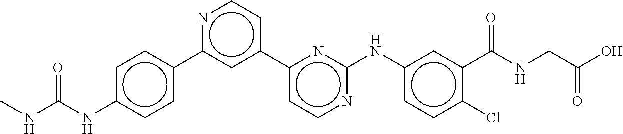 |
| 19 | 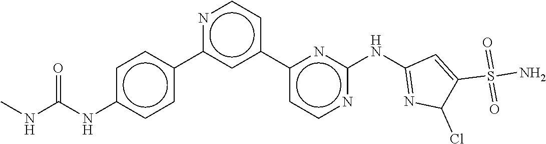 |
| 20 | 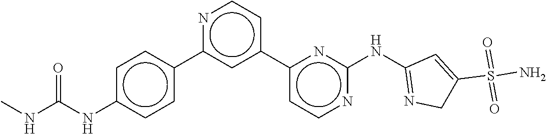 |
| 21 | 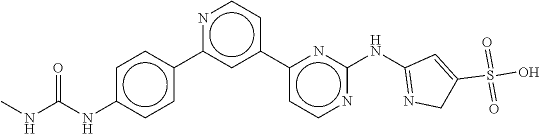 |
| 22 | 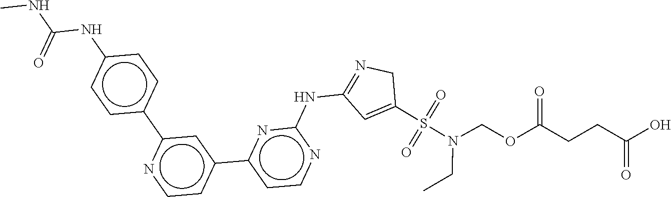 |
| 23 | 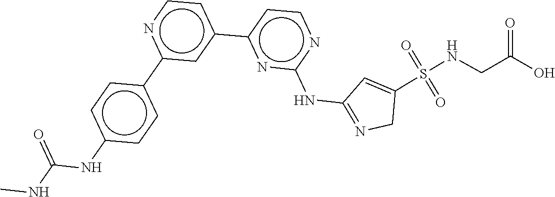 |
| 24 | 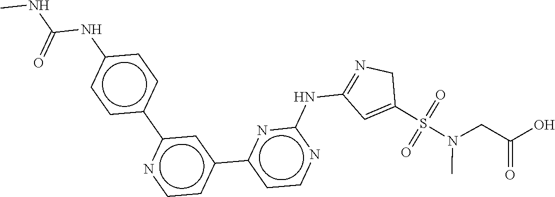 |
| 25 | 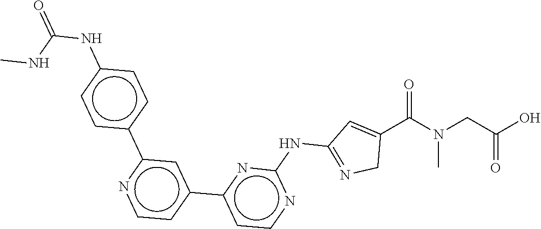 |
| 26 | 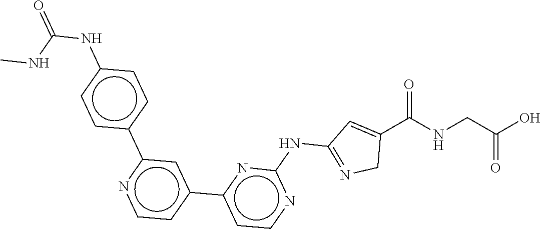 |
| 27 | 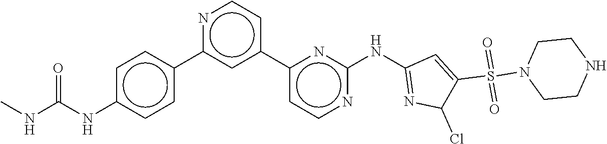 |
| 28 | 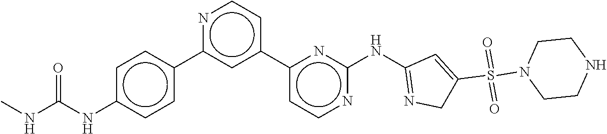 |
| 29 | 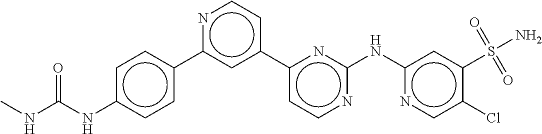 |
| 30 | 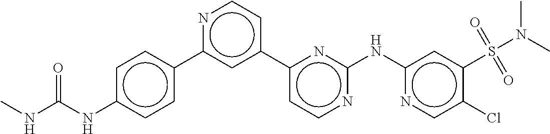 |
| 31 | 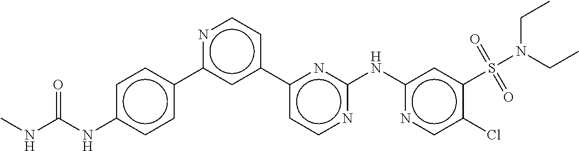 |
| 32 | 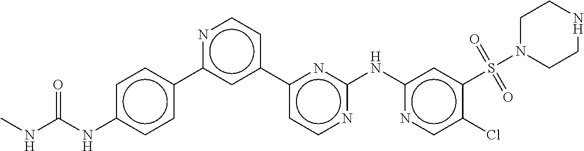 |

TABLE 2

| Compound | Data - 1 | | Data-2 | |
|---|---|---|---|---|
| 1 | miLogP | 3.32 | | |
| | TPSA | 141.24 | | |
| | natoms | 40 | | |
| | MW | 579.09 | | |
| | nON | 11 | | |
| | nOHNH | 4 | | |
| | nviolations | 2 | | |
| | nrotb | 7 | | |
| | volume | 485.07 | | |
| 2 | miLogP | 2.76 | | |
| | TPSA | 169.66 | | |
| | natoms | 41 | | |
| | MW | 598.09 | | |
| | nON | 12 | | |
| | nOHNH | 5 | | |
| | nviolations | 2 | | |
| | nrotb | 11 | | |
| | volume | 499.55 | | |
| 3 | miLogP | 2.74 | | |
| | TPSA | 169.66 | | |
| | natoms | 39 | | |
| | MW | 570.03 | | |
| | nON | 12 | | |
| | nOHNH | 5 | | |
| | nviolations | 2 | | |
| | nrotb | 9 | | |
| | volume | 465.94 | | |
| 4 | miLogP | 3.68 | | |
| | TPSA | 143.80 | | |
| | natoms | 39 | | |
| | MW | 561.03 | | |
| | nON | 11 | | |
| | nOHNH | 3 | | |
| | nviolations | 2 | | |
| | nrotb | 7 | | |
| | volume | 456.14 | | |
| 5 | miLogP | 3.53 | | |
| | TPSA | 149.35 | | |
| | natoms | 35 | | |
| | MW | 510.88 | | |
| | nON | 10 | | |
| | nOHNH | 5 | | |
| | nviolations | 1 | | |
| | nrotb | 6 | | |
| | volume | 416.47 | | |
| 6 | miLogP | 3.54 | | |
| | TPSA | 143.80 | | |
| | natoms | 40 | | |
| | MW | 554.64 | | |
| | nON | 11 | | |
| | nOHNH | 3 | | |
| | nviolations | 2 | | |
| | nrotb | 7 | | |
| | volume | 475.73 | | |
| 7 | miLogP | 3.90 | | |
| | TPSA | 132.45 | | |
| | natoms | 40 | | |
| | MW | 579.09 | | |
| | nON | 11 | | |
| | nOHNH | 3 | | |
| | nviolations | 2 | | |
| | nrotb | 7 | | |
| | volume | 485.21 | | |
| 8 | miLogP | 2.74 | | |
| | TPSA | 166.43 | | |
| | natoms | 39 | | |
| | MW | 569.04 | | |
| | nON | 11 | | |
| | nOHNH | 5 | | |
| | nviolations | 2 | | |
| | nrotb | 9 | | |
| | volume | 469.99 | | |
| 9 | miLogP | 2.98 | | |
| | TPSA | 124.16 | | |
| | natoms | 39 | | |
| | MW | 543.03 | | |
| | nON | 10 | | |
| | nOHNH | 4 | | |
| | nviolations | 1 | | |
| | nrotb | 6 | | |
| | volume | 472.62 | | |
| 10 | miLogP | 2.75 | | |
| | TPSA | 124.16 | | |
| | natoms | 39 | | |
| | MW | 522.61 | | |
| | nON | 10 | | |
| | nOHNH | 4 | | |
| | nviolations | 1 | | |
| | nrotb | 6 | | |
| | volume | 475.65 | | |
| 11 | miLogP | 3.92 | | |
| | TPSA | 132.45 | | |
| | natoms | 41 | | |
| | MW | 593.11 | | |
| | nON | 11 | | |
| | nOHNH | 3 | | |
| | nviolations | 2 | | |
| | nrotb | 7 | | |
| | volume | 502.01 | | |
| 12 | miLogP | 3.41 | olinspiration bioactivity | |
| | TPSA | 151.99 | score v2022.08 | |
| | natoms | 35 | GPCR ligand | 0.07 |
| | MW | 509.98 | Ion channel modulator | 0.02 |
| | nON | 10 | Kinase inhibitor | 0.57 |
| | nOHNH | 5 | Nuclear receptor ligand | −0.53 |
| | nviolations | 1 | Protease inhibitor | −0.01 |
| | nrotb | 6 | Enzyme inhibitor | 0.23 |
| | volume | 414.81 | | |
| 13 | miLogP | 1.70 | Molinspiration bioactivity | |
| | TPSA | 146.20 | score v2022.08 | |
| | natoms | 35 | GPCR ligand | 0.22 |
| | MW | 510.96 | Ion channel modulator | 0.12 |
| | nON | 10 | Kinase inhibitor | 0.50 |
| | nOHNH | 4 | Nuclear receptor ligand | −0.55 |
| | nviolations | 1 | Protease inhibitor | 0.05 |
| | nrotb | 6 | Enzyme inhibitor | 0.22 |
| | volume | 411.54 | | |
| 14 | miLogP | 3.73 | Molinspiration bioactivity | |
| | TPSA | 192.81 | score v2022.08 | |
| | natoms | 46 | GPCR ligand | −0.14 |
| | MW | 668.13 | Ion channel modulator | −0.75 |
| | nON | 14 | Kinase inhibitor | −0.22 |
| | nOHNH | 4 | Nuclear receptor ligand | −0.83 |
| | nviolations | 2 | Protease inhibitor | 0.03 |
| | nrotb | 14 | Enzyme inhibitor | −0.36 |
| | volume | 555.04 | | |
| 15 | miLogP | 2.11 | Molinspiration bioactivity | |
| | TPSA | 175.3 | score v2022.08 | |
| | natoms | 39 | GPCR ligand | 0.15 |
| | MW | 568.01 | Ion channel modulator | −0.17 |
| | nON | 12 | Kinase inhibitor | 0.34 |
| | nOHNH | 5 | Nuclear receptor ligand | −0.38 |
| | nviolations | 2 | Protease inhibitor | 0.01 |
| | nrotb | 9 | Enzyme inhibitor | 0.16 |
| | volume | 459.73 | | |
| 16 | miLogP | 2.36 | Molinspiration bioactivity | |
| | TPSA | 166.51 | score v2022.08 | |
| | natoms | 40 | GPCR ligand | 0.14 |
| | MW | 582.04 | Ion channel modulator | −0.26 |
| | nON | 12 | Kinase inhibitor | 0.24 |
| | nOHNH | 4 | Nuclear receptor ligand | −0.49 |
| | nviolations | 2 | Protease inhibitor | 0.02 |
| | nrotb | 9 | Enzyme inhibitor | 0.07 |
| | volume | 476.67 | | |
| 17 | miLogP | 2.01 | Molinspiration bioactivity | |
| | TPSA | 149.44 | score v2022.08 | |
| | natoms | 39 | GPCR ligand | 0.22 |
| | MW | 545.99 | Ion channel modulator | −0.07 |
| | nON | 11 | Kinase inhibitor | 0.48 |
| | nOHNH | 4 | Nuclear receptor ligand | −0.28 |
| | nviolations | 2 | Protease inhibitor | 0.01 |
| | nrotb | 8 | Enzyme inhibitor | 0.12 |
| | volume | 464.22 | | |
| 18 | miLogP | 2.24 | Molinspiration bioactivity | |
| | TPSA | 158.22 | score v2022.08 | |
| | natoms | 38 | GPCR ligand | 0.18 |
| | MW | 531.96 | Ion channel modulator | −0.01 |
| | nON | 11 | Kinase inhibitor | 0.50 |
| | nOHNH | 5 | Nuclear receptor ligand | −0.28 |
| | nviolations | 2 | Protease inhibitor | 0.02 |
| | nrotb | 8 | Enzyme inhibitor | 0.15 |
| | volume | 447.28 | | |
| 19 | miLogP | 1.79 | Molinspiration bioactivity | |
| | TPSA | 164.36 | score v2022.08 | |
| | natoms | 34 | GPCR ligand | 0.19 |
| | MW | 498.96 | Ion channel modulator | 0.06 |
| | nON | 11 | Kinase inhibitor | 0.40 |
| | nOHNH | 5 | Nuclear receptor ligand | −0.25 |
| | nviolations | 1 | Protease inhibitor | 0.05 |
| | nrotb | 6 | Enzyme inhibitor | 0.30 |
| | volume | 400.06 | | |
| 20 | miLogP | 1.21 | Molinspiration bioactivity | |
| | TPSA | 164.36 | score v2022.08 | |
| | natoms | 33 | GPCR ligand | 0.22 |
| | MW | 464.51 | Ion channel modulator | 0.15 |
| | nON | 11 | Kinase inhibitor | 0.47 |
| | nOHNH | 5 | Nuclear receptor ligand | −0.19 |
| | nviolations | 1 | Protease inhibitor | 0.14 |
| | nrotb | 6 | Enzyme inhibitor | 0.32 |
| | volume | 386.50 | | |
| 21 | miLogP | −0.49 | Molinspiration bioactivity | |
| | TPSA | 158.56 | score v2022.08 | |
| | natoms | 33 | GPCR ligand | 0.22 |
| | MW | 465.50 | Ion channel modulator | 0.13 |
| | nON | 11 | Kinase inhibitor | 0.46 |
| | nOHNH | 4 | Nuclear receptor ligand | −0.16 |
| | nviolations | 1 | Protease inhibitor | 0.10 |
| | nrotb | 6 | Enzyme inhibitor | 0.32 |
| | volume | 383.23 | | |
| 22 | Molinspiration property | | Molinspiration bioactivity | |
| | engine v2022.08 | | score v2022.08 | |
| | miLogP | 1.53 | GPCR ligand | 0.05 |
| | TPSA | 205.18 | Ion channel modulator | −0.40 |
| | natoms | 44 | Kinase inhibitor | −0.05 |
| | MW | 622.66 | Nuclear receptor ligand | −0.46 |
| | nON | 15 | Protease inhibitor | 0.19 |
| | nOHNH | 4 | Enzyme inhibitor | −0.15 |
| | nviolations | 2 | | |
| | nrotb | 14 | | |
| | volume | 526.73 | | |
| 23 | Molinspiration property | | Molinspiration bioactivity | |
| | engine v2022.08 | | score v2022.08 | |
| | miLogP | −0.08 | GPCR ligand | 0.25 |
| | TPSA | 187.66 | Ion channel modulator | 0.05 |
| | natoms | 37 | Kinase inhibitor | 0.30 |
| | MW | 522.55 | Nuclear receptor ligand | −0.12 |
| | nON | 13 | Protease inhibitor | 0.17 |
| | nOHNH | 5 | Enzyme inhibitor | 0.27 |
| | nviolations | 2 | | |
| | nrotb | 9 | | |
| | volume | 431.42 | | |
| 24 | Molinspiration property | | Molinspiration bioactivity | |
| | engine v2022.08 | | score v2022.08 | |
| | miLogP | 0.16 | GPCR ligand | 0.22 |
| | TPSA | 178.87 | Ion channel modulator | 0.13 |
| | natoms | 38 | Kinase inhibitor | 0.36 |
| | MW | 536.57 | Nuclear receptor ligand | −0.11 |
| | nON | 13 | Protease inhibitor | 0.12 |
| | nOHNH | 4 | Enzyme inhibitor | 0.21 |
| | nviolations | 2 | | |
| | nrotb | 9 | | |
| | volume | 448.36 | | |
| 25 | Molinspiration property | | Molinspiration bioactivity | |
| | engine v2022.08 | | score v2022.08 | |
| | miLogP | 0.32 | GPCR ligand | 0.22 |
| | TPSA | 161.80 | Ion channel modulator | 0.13 |
| | natoms | 37 | Kinase inhibitor | 0.36 |
| | MW | 500.52 | Nuclear receptor ligand | −0.11 |
| | nON | 12 | Protease inhibitor | 0.12 |
| | nOHNH | 4 | Enzyme inhibitor | 0.21 |
| | nviolations | 2 | | |
| | nrotb | 8 | | |
| | volume | 435.91 | | |
| 26 | Molinspiration property | | Molinspiration bioactivity | |
| | engine v2022.08 | | score v2022.08 | |
| | miLogP | 0.07 | GPCR ligand | 0.22 |
| | TPSA | 170.59 | Ion channel modulator | 0.12 |
| | natoms | 36 | Kinase inhibitor | 0.34 |
| | MW | 486.49 | Nuclear receptor ligand | −0.18 |
| | nON | 12 | Protease inhibitor | 0.10 |
| | nOHNH | 5 | Enzyme inhibitor | 0.22 |
| | nviolations | 1 | | |
| | nrotb | 8 | | |
| | volume | 418.97 | | |
| 27 | miLogP | 1.71 | Molinspiration bioactivity | |
| | TPSA | 153.60 | score v2022.08 | |
| | natoms | 39 | GPCR ligand | 0.18 |
| | MW | 568.06 | Ion channel modulator | −0.21 |
| | nON | 12 | Kinase inhibitor | 0.34 |
| | nOHNH | 4 | Nuclear receptor ligand | −0.32 |
| | nviolations | 2 | Protease inhibitor | 0.03 |
| | nrotb | 7 | Enzyme inhibitor | 0.10 |
| | volume | 470.32 | | |
| 28 | Molinspiration property | | Molinspiration bioactivity | |
| | engine v2022.08 | | score v2022.08 | |
| | miLogP | 1.13 | GPCR ligand | 0.20 |
| | TPSA | 153.60 | Ion channel modulator | −0.09 |
| | natoms | 38 | Kinase inhibitor | 0.42 |
| | MW | 533.62 | Nuclear receptor ligand | −0.25 |
| | nON | 12 | Protease inhibitor | 0.09 |
| | nOHNH | 4 | Enzyme inhibitor | 0.14 |
| | nviolations | 2 | | |
| | nrotb | 7 | | |
| | volume | 456.76 | | |
| 29 | Molinspiration property | | Molinspiration bioactivity | |
| | engine v2022.08 | | score v2022.08 | |
| | miLogP | 2.68 | GPCR ligand | 0.24 |
| | TPSA | 164.88 | Ion channel modulator | 0.09 |
| | natoms | 35 | Kinase inhibitor | 0.70 |
| | MW | 510.97 | Nuclear receptor ligand | −0.41 |
| | nON | 11 | Protease inhibitor | 0.08 |
| | nOHNH | 5 | Enzyme inhibitor | 0.37 |
| | nviolations | 2 | | |
| | nrotb | 6 | | |
| | volume | 410.65 | | |
| 30 | Molinspiration property | | Molinspiration bioactivity | |
| | engine v2022.08 | | score v2022.08 | |
| | miLogP | 3.30 | GPCR ligand | 0.22 |
| | TPSA | 142.10 | Ion channel modulator | −0.06 |
| | natoms | 37 | Kinase inhibitor | 0.53 |
| | MW | 539.02 | Nuclear receptor ligand | −0.39 |
| | nON | 11 | Protease inhibitor | −0.02 |
| | nOHNH | 3 | Enzyme inhibitor | 0.18 |
| | nviolations | 2 | | |
| | nrotb | 7 | | |
| | volume | 445.27 | | |
| 31 | Molinspiration property | | Molinspiration bioactivity | |
| | engine v2022.08 | | score v2022.08 | |
| | miLogP | 4.05 | GPCR ligand | 0.26 |
| | TPSA | 142.10 | Ion channel modulator | −0.22 |
| | natoms | 39 | Kinase inhibitor | 0.47 |
| | MW | 567.08 | Nuclear receptor ligand | −0.40 |
| | nON | 11 | Protease inhibitor | 0.06 |
| | nOHNH | 3 | Enzyme inhibitor | 0.15 |
| | nviolations | 2 | | |
| | nrotb | 9 | | |
| | volume | 478.87 | | |
| 32 | miLogP | 2.59 | Molinspiration bioactivity | |
| | TPSA | 154.13 | score v2022.08 | |
| | natoms | 40 | GPCR ligand | 0.26 |
| | MW | 580.07 | Ion channel modulator | −0.22 |
| | nON | 12 | Kinase inhibitor | 0.47 |
| | nOHNH | 4 | Nuclear receptor ligand | −0.40 |
| | nviolations | 2 | Protease inhibitor | 0.06 |
| | nrotb | 7 | Enzyme inhibitor | 0.15 |
| | volume | 480.91 | | |

What is claimed is:

1. A compound of Formula I 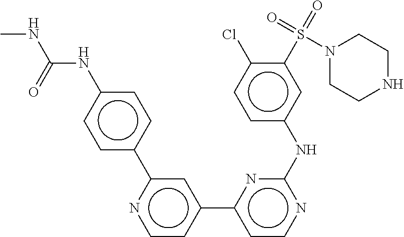
or a pharmaceutically acceptable salt thereof.

2. A pharmaceutical composition comprising:
(a) the compound of Formula I or a pharmaceutically acceptable salt thereof; and
(b) a pharmaceutically acceptable carrier.

3. The pharmaceutical composition of claim 2, wherein the compound of Formula I inhibits nonsense mediated decay (NMD).

4. The pharmaceutical composition of claim 2 or 3, wherein the cell-surface expression of a neoantigen is increased.

5. The pharmaceutical composition of claim 4, wherein the cell-surface expression is presentation of the neoantigen on a MHC class I HLA molecule.

6. The pharmaceutical composition of claim 4, wherein the neoantigen is derived from a TP53 (tumor protein p53) gene, EXOC1 gene, SPTAN1 gene, RAB14 gene, or ZDHHC16 gene.

7. A method of inhibiting nonsense mediated decay (NMD), the method comprising delivering the compound of Formula I into a cell, thereby inhibiting NMD in the cell.

8. The method of claim 7, wherein the cell-surface expression of a neoantigen is increased.

9. The method of claim 8, the cell-surface expression is presentation of the neoantigen on a MHC class I HLA molecule.

10. The method of claim 8, wherein the neoantigen is derived from a TP53 (tumor protein p53) gene, EXOC1 gene, SPTAN1 gene, RAB14 gene, or ZDHHC16 gene.

11. A method of enhancing immunotherapy in a subject in need thereof, the method comprising administering to the subject the compound of claim 1, or the pharmaceutical composition of any one of claims 2-6.

12. The method of claim 11, wherein the compound of claim 1, or the pharmaceutical composition of any one of claims 2-6 inhibits nonsense mediated decay (NMD).

13. The method of claim 11 or 12, wherein cell-surface expression of a neoantigen is increased in the subject following administration.

14. The method of claim 13, the cell-surface expression is presentation of the neoantigen on a MHC class I HLA molecule

15. The method of claim 13, wherein the neoantigen is derived from a TP53 (tumor protein p53) gene, EXOC1 gene, SPTAN1 gene, RAB14 gene, or ZDHHC16 gene.

16. The method of any one of claims 11-15, wherein the subject has been identified or diagnosed as having a cancer.

17. The method of claim 16, wherein the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

18. A method of treating cancer in a subject in need thereof, the method comprising administering to the subject:
(a) the compound of claim 1, or the pharmaceutical composition of any one of claims 2-6; and
(b) an immunotherapy.

19. The method of claim 18, wherein the immunotherapy comprises an immune checkpoint inhibitor, an oncolytic virus therapy, a cell-based therapy, and a cancer vaccine.

20. The method of claim 18 or 19, wherein the method further comprises administering to the subject one or more additional anticancer therapies.

21. The method of claim 20, wherein the one or more additional anticancer therapies comprise a chemotherapeutic agent, ionizing radiation, a therapeutic antibody, or gene therapy.

22. The method of any one of claims 18-21, wherein the compound of claim 1, or the pharmaceutical composition of any one of claims 2-6 inhibits nonsense mediated decay (NMD).

23. The method of any one of claims 18-22, wherein the cell-surface expression of a neoantigen is increased in the subject following administration.

24. The method of claim 23, the cell-surface expression is presentation of the neoantigen on a MHC class I HLA molecule.

25. The method of claim 23, wherein the neoantigen is derived from a TP53 (tumor protein p53) gene, EXOC1 gene, SPTAN1 gene, RAB14 gene, or ZDHHC16 gene.

26. The method of any one of claims 18-25, wherein the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

27. A compound of Formula II: 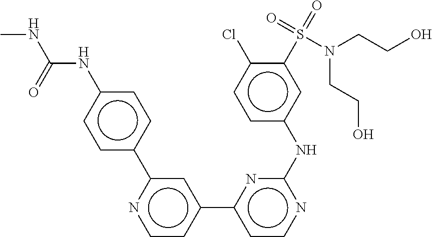
or a pharmaceutically acceptable salt thereof.

28. A pharmaceutical composition comprising:
(a) the compound of Formula II or a pharmaceutically acceptable salt thereof; and
(b) a pharmaceutically acceptable carrier.

29. The pharmaceutical composition of claim 28, wherein the compound of Formula II inhibits nonsense mediated decay (NMD).

30. The pharmaceutical composition of claim 28 or 29, wherein the cell-surface expression of a neoantigen is increased.

31. The pharmaceutical composition of claim 30, wherein the cell-surface expression is presentation of the neoantigen on a MHC class I HLA molecule.

32. The pharmaceutical composition of claim 30, wherein the neoantigen is derived from a TP53 (tumor protein p53) gene, EXOC1 gene, SPTAN1 gene, RAB14 gene, or ZDHHC16 gene.

33. A method of inhibiting nonsense mediated decay (NMD), the method comprising delivering the compound of Formula II into a cell, thereby inhibiting NMD in the cell.

34. The method of claim 33, wherein the cell-surface expression of a neoantigen is increased.

35. The method of claim 34, the cell-surface expression is presentation of the neoantigen on a MHC class I HLA molecule.

36. The method of claim 34, wherein the neoantigen is derived from a TP53 (tumor protein p53) gene, EXOC1 gene, SPTAN1 gene, RAB14 gene, or ZDHHC16 gene.

37. A method of enhancing immunotherapy in a subject in need thereof, the method comprising administering to the subject the compound of claim 27, or the pharmaceutical composition of any one of claims 28-32.

38. The method of claim 37, wherein the compound of claim 27, or the pharmaceutical composition of any one of claims 28-32 inhibits nonsense mediated decay (NMD).

39. The method of claim 37 or 38, wherein cell-surface expression of a neoantigen is increased in the subject following administration.

40. The method of claim 39, the cell-surface expression is presentation of the neoantigen on a MHC class I HLA molecule

41. The method of claim 39, wherein the neoantigen is derived from a TP53 (tumor protein p53) gene, EXOC1 gene, SPTAN1 gene, RAB14 gene, or ZDHHC16 gene.

42. The method of any one of claims 37-41, wherein the subject has been identified or diagnosed as having a cancer.

43. The method of claim 42, wherein the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

44. A method of treating cancer in a subject in need thereof, the method comprising administering to the subject:
(a) the compound of claim 27, or the pharmaceutical composition of any one of claims 28-32; and
(b) an immunotherapy.

45. The method of claim 44, wherein the immunotherapy comprises an immune checkpoint inhibitor, an oncolytic virus therapy, a cell-based therapy, and a cancer vaccine.

46. The method of claim 44 or 45, wherein the method further comprises administering to the subject one or more additional anticancer therapies.

47. The method of claim 46, wherein the one or more additional anticancer therapies comprise a chemotherapeutic agent, ionizing radiation, a therapeutic antibody, or gene therapy.

48. The method of any one of claims 44-47, wherein the compound of claim 27, or the pharmaceutical composition of any one of claims 28-32 inhibits nonsense mediated decay (NMD).

49. The method of any one of claims 44-48, wherein the cell-surface expression of a neoantigen is increased in the subject following administration.

50. The method of claim 49, the cell-surface expression is presentation of the neoantigen on a MHC class I HLA molecule.

51. The method of claim 49, wherein the neoantigen is derived from a TP53 (tumor protein p53) gene, EXOC1 gene, SPTAN1 gene, RAB14 gene, or ZDHHC16 gene.

52. The method of any one of claims 44-51, wherein the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

53. A compound of Formula III: 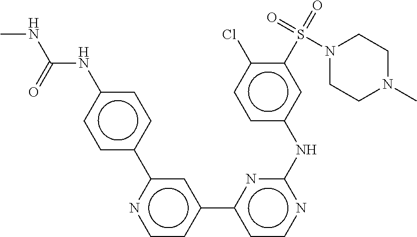
or a pharmaceutically acceptable salt thereof.

54. A pharmaceutical composition comprising:
(a) the compound of Formula III or a pharmaceutically acceptable salt thereof; and
(b) a pharmaceutically acceptable carrier.

55. The pharmaceutical composition of claim 54, wherein the compound of Formula III inhibits nonsense mediated decay (NMD).

56. The pharmaceutical composition of claim 54 or 55, wherein the cell-surface expression of a neoantigen is increased.

57. The pharmaceutical composition of claim 56, wherein the cell-surface expression is presentation of the neoantigen on a MHC class I HLA molecule.

58. The pharmaceutical composition of claim 56, wherein the neoantigen is derived from a TP53 (tumor protein p53) gene, EXOC1 gene, SPTAN1 gene, RAB14 gene, or ZDHHC16 gene.

59. A method of inhibiting nonsense mediated decay (NMD), the method comprising delivering the compound of Formula III into a cell, thereby inhibiting NMD in the cell.

60. The method of claim 59, wherein the cell-surface expression of a neoantigen is increased.

61. The method of claim 60, the cell-surface expression is presentation of the neoantigen on a MHC class I HLA molecule.

62. The method of claim 60, wherein the neoantigen is derived from a TP53 (tumor protein p53) gene, EXOC1 gene, SPTAN1 gene, RAB14 gene, or ZDHHC16 gene.

63. A method of enhancing immunotherapy in a subject in need thereof, the method comprising administering to the subject the compound of claim 53, or the pharmaceutical composition of any one of claims 54-58.

64. The method of claim 63, wherein the compound of claim 53, or the pharmaceutical composition of any one of claims 54-58 inhibits nonsense mediated decay (NMD).

65. The method of claim 63 or 64, wherein cell-surface expression of a neoantigen is increased in the subject following administration.

66. The method of claim 65, the cell-surface expression is presentation of the neoantigen on a MHC class I HLA molecule

67. The method of claim 65, wherein the neoantigen is derived from a TP53 (tumor protein p53) gene, EXOC1 gene, SPTAN1 gene, RAB14 gene, or ZDHHC16 gene.

68. The method of any one of claims 63-67, wherein the subject has been identified or diagnosed as having a cancer.

69. The method of claim 68, wherein the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

70. A method of treating cancer in a subject in need thereof, the method comprising administering to the subject:
(a) the compound of claim 53, or the pharmaceutical composition of any one of claims 54-58; and
(b) an immunotherapy.

71. The method of claim 70, wherein the immunotherapy comprises an immune checkpoint inhibitor, an oncolytic virus therapy, a cell-based therapy, and a cancer vaccine.

72. The method of claim 70 or 71, wherein the method further comprises administering to the subject one or more additional anticancer therapies.

73. The method of claim 72, wherein the one or more additional anticancer therapies comprise a chemotherapeutic agent, ionizing radiation, a therapeutic antibody, or gene therapy.

74. The method of any one of claims 70-73, wherein the compound of claim 53, or the pharmaceutical composition of any one of claims 54-58 inhibits nonsense mediated decay (NMD).

75. The method of any one of claims 70-74, wherein the cell-surface expression of a neoantigen is increased in the subject following administration.

76. The method of claim 75, the cell-surface expression is presentation of the neoantigen on a MHC class I HLA molecule.

77. The method of claim 75, wherein the neoantigen is derived from a TP53 (tumor protein p53) gene, EXOC1 gene, SPTAN1 gene, RAB14 gene, or ZDHHC16 gene.

78. The method of any one of claims 70-77, wherein the cancer is a solid tumor, microsatellite-instability high (MSI-high) tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, lung squamous cell carcinoma, head and neck carcinoma, endometrial cancer, cervical cancer, liver cancer, or hepatocellular carcinoma.

79. A compound having formula (V): 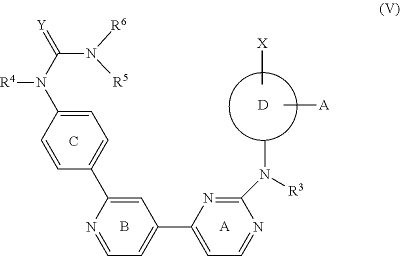
or a pharmaceutically acceptable salt thereof; wherein:
ring D is
phenyl;
heteroaryl of 5-6 ring atoms, wherein 1-3 ring atoms are heteroatoms, each independently selected from the group consisting of N, N(R⁷), O, and S; and wherein R⁷ is H, C₁₋₃ alkyl, or —C(O)C₁₋₃ alkyl; or
heterocyclyl or heterocycloalkenyl of 4-7 ring atoms, wherein from 1-3 ring atoms are heteroatoms, each independently selected from the group consisting of N, N(R⁷), O, and S; and wherein R⁷ is H, C₁₋₃ alkyl, or —C(O)C₁₋₃ alkyl;
X is H, —F, Cl, C₁-C₄ alkyl, or CN;
A is —SO₂NR¹R², —C(O)NR¹R², —SO₃H, —SO₂R^{A}, or —PO(OH)₂;
R^{A} is C₁₋₆ alkyl, which is optionally substituted with 1-3 substituents independently selected from the group consisting of: —OH, C₁₋₄ alkoxy, C₁₋₄ haloalkoxy, and F;
each occurrence of R¹ and R² is independently selected from the group consisting of: H and C₁₋₆ alkyl, wherein the C₁₋₆ alkyl is optionally substituted with 1-3 substituents independently selected from the group consisting of:
—CO₂H;
—CO₂(C₁₋₄ alkyl);
—OC(O)(CH₂)ₙ—CO₂H, wherein n is 1-4 (e.g., n is 2);
—OC(O)(CH₂)ₙ—CO₂(C₁₋₄ alkyl), wherein n is 1-4 (e.g., n is 2);
—OH;
C₁₋₄ alkoxy;
C₁₋₄ haloalkoxy;
—F, and
—NR′R″, wherein each of R′ and R′ is independently selected from the group consisting of H and C₁₋₄ alkyl;
or
R¹ and R², taken together with the nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of N, N(R⁷), O, and S; and wherein the ring is optionally substituted with 1-4 independently selected R^{a}; wherein R⁷ is H, C₁₋₆ alkyl, or —C(O)C₁₋₆ alkyl;
each of rings A, B, and C is independently and optionally substituted with 1-3 independently selected R^{a};
each occurrence of R³ and R⁴ is independently selected from the group consisting of: H and C₁₋₃ alkyl;
each occurrence of R⁵ and R⁶ is independently selected from the group consisting of: H and C₁₋₆ alkyl,
Y is O or S;
each occurrence of R^{a} is independently selected from the group consisting of halo, C₁₋₆ alkyl, C₁₋₆ haloalkyl, cyano, C₁₋₆ alkoxy, C₁₋₆ haloalkoxy, C₁₋₆ thioalkoxy, C₁₋₆ halothioalkoxy, S(O)₂(C₁₋₆ alkyl), C₃₋₆ cycloalkyl, C₆₋₁₀ aryl, NR⁸R⁹, C(O)R⁸, and C(O)NR⁸R⁹, and
each occurrence of R⁸ and R⁹ is independently selected from the group consisting of H and C₁₋₃ alkyl.

80. The compound of claim 79, wherein ring D is phenyl.

81. The compound of claim 79 or 80, wherein ring D has the formula:

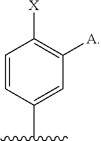

82. The compound of claim 79, wherein ring D is heteroaryl of 5-6 ring atoms, wherein from 1-3 ring atoms are heteroatoms, each independently selected from the group consisting of N, N(R⁷), O, and S; and wherein R⁷ is H, C₁₋₃ alkyl, or —C(O)C₁₋₃ alkyl.

83. The compound of claim 79 or 82, wherein ring D is heteroaryl of 5 ring atoms, wherein from 1-3 ring atoms are heteroatoms, each independently selected from the group consisting of N, N(R⁷), O, and S; and wherein R⁷ is H, C₁₋₃ alkyl, or —C(O)C₁₋₃ alkyl.

84. The compound of claim 79, 82, or 83, wherein ring D is 2H-pyrrolyl.

85. The compound of any one of claims 79 and 82-84, wherein ring D is:

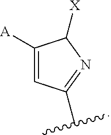

86. The compound of claim 79 or 82, wherein ring D is heteroaryl of 6 ring atoms, wherein from 1-3 ring atoms are heteroatoms, each independently selected from the group consisting of N, N(R⁷), O, and S; and wherein R⁷ is H, C₁₋₃ alkyl, or —C(O)C₁₋₃ alkyl.

87. The compound of claim 79, 82, or 86, wherein ring D is pyridyl.

88. The compound of claim 79, 82, 86, or 87, wherein ring D is:

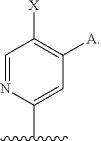

89. The compound of any one of claims 79-88, wherein A is —SO₂NR¹R².

90. The compound of claim 89, wherein each occurrence of R¹ and R² is H.

91. The compound of claim 89, wherein each occurrence of R¹ and R² is C₁₋₆ alkyl that is optionally substituted.

92. The compound of claim 89, wherein R¹ and R², taken together with the nitrogen atom connecting them, forms a saturated, partially unsaturated, or aromatic ring including 4-8 ring atoms, wherein from 0-2 ring atoms (in addition to the nitrogen atom connecting R¹ and R²) are ring heteroatoms each independently selected from the group consisting of: N, N(R⁷), O, and S; and wherein the ring is optionally substituted with 1-4 independently selected R^{a}; wherein R⁷ is H, C₁₋₆ alkyl, or —C(O)C₁₋₆ alkyl.

93. The compound of any one of claims 79-92, wherein X is Cl.
